(19) United States
(12) Patent Application Publication
(10) Pub. No.: US 2026/0002204 A1
(43) Pub. Date: Jan. 1, 2026

(54) METHOD FOR PRODUCING SPACER-SPECIFIC DNA RECOMBINING ENZYMES

(71) Applicant: TECHNISCHE UNIVERSITÄT DRESDEN KÖRPERSCHAFT DES ÖFFENTLICHEN RECHTS, Dresden (DE)

(72) Inventors: Frank Buchholz, Dresden (DE); Jenna Hoersten, Dresden (DE); Felix Lansing, Nürnberg (DE); Maciej Paszkowski-Rogacz, Dresden (DE)

(21) Appl. No.: 19/230,463

(22) Filed: Jun. 6, 2025

(30) Foreign Application Priority Data
Jun. 7, 2024 (EP) 24180926.8

(51) Int. Cl. C12Q 1/686; A61K 31/7088; A61K 48/00; C12N 9/00; C12N 15/52; C12N 15/63; C12Q 1/48; C12Q 1/6813; C12Q 1/6876

(52) U.S. Cl. CPC C12Q 1/686; A61K 31/7088; A61K 48/005; C12N 9/00; C12N 15/52; C12N 15/63; C12Q 1/48; C12Q 1/6813; C12Q 1/6876; C12Y 207/07

(57) ABSTRACT

The present invention pertains to a method for generating a spacer-specific DNA recombining enzyme (DRE), the method comprising the steps of: a) providing a library of expression vectors encoding a plurality of variants of a DRE (vDRE), wherein the amino acid sequences of the vDREs comprise one or more amino acid modifications in comparison to a non-variant DRE (nvDRE) from which the vDREs are derived, wherein the nvDRE binds to a first target site comprising in 5′ to 3′ direction a half-site A (HSA), a spacer (Spacer) and a half-site B (HSB), wherein each expression vector comprises: (i) a first region comprising a nucleotide sequence encoding one of the vDREs from among the plurality of vDREs operably linked to an expression control sequence, and (ii) a second region comprising a nucleotide sequence comprising in 5′ to 3′ direction a first target site, an insert nucleotide sequence (INS) of a length at least 1 nucleotide and a second target site, wherein each of the first and second target site comprises the HSA, a variant spacer (vSpacer) and the HSB, wherein the vSpacer differs by at least one nucleotide from the Spacer; b) introducing the library of expression vectors into host cells; c) expressing the plurality of vDREs in the host cells; d) optionally isolating DNA from the host cells; and e) determining whether the vDRE shows activity on the target sites of at least one expression vector.

### RELATED APPLICATIONS

This application claims priority to European Patent Application No. 24180926.8, filed Jun. 7, 2024, the entire disclosure of which is hereby incorporated herein by reference.

### SEQUENCE LISTING

The instant application contains a Sequence Listing which has been submitted electronically in XML format and is hereby incorporated by reference in its entirety. Said XML file, created on May 27, 2025, is named 765732_TUD9-008_ST26.xml and is 22,350 bytes in size.

### FIELD OF THE INVENTION

The present invention relates to a method for producing a spacer-specific DNA recombining enzyme. The present invention further pertains to spacer-specific DNA recombining enzymes obtained by said method, and to systems comprising the same, nucleic acids encoding the same, vectors expressing the nucleic acids, as well as uses of said spacer-specific DNA recombining enzymes for performing a recombination in a nucleic acid sequence, preferably in the genome of a subject or a cell.

### BACKGROUND OF THE INVENTION

Genome integration, the process of introducing foreign DNA into the genome of an organism, is a fundamental technique in molecular biology and genetic engineering. In the realms of medicine and biotechnology, the ability to precisely manipulate genomes is of paramount importance for advancements in therapeutics, disease research, and biopharmaceutical production.

One crucial aspect of genome editing is the site-specific integration of genetic payloads into desired genomic loci. Integrating sizable genetic payloads at precise genomic locations offers numerous advantages, such as targeted gene therapy, precise gene regulation, and the creation of genetically modified organisms for various research and industrial purposes. Targeted integration allows the user to insert the transgene into a locus that favors stable, long-term expression. It helps to avoid strong positional effects and reduce unwanted effects on cell functions, as well as to control the copy numbers at each integration event. However, achieving target site-specific integration of large DNA fragments remains a significant challenge in current genome editing methodologies, currently requiring inefficient, multi-step processes that are time and resource intensive (Anzalone et al. 2022; Yarnall et al. 2023; Zhu et al. 2014).

Site-specific recombinases (SSRs) are specialized enzymes that promote site-specific DNA rearrangements between defined target regions (Grindley et al., 2006). Engineering and directed evolution strategies have been successfully used to alter the site-specificity of recombinases, allowing SSRs to be an adaptable tool for precise genome engineering (Meinke et al., 2016). Of particular interest are the tailor-made recombinase systems derived from the Cre/loxP system, where the native DNA specificity is altered to enable the recombination of therapeutically relevant sequences (Lansing et al., 2020; Buchholz et al., 2001; Lansing et al., 2022; Karpinski et al., 2016; Sarkar et al., 2007; Rojo-Romanos et al., 2023; Abi-Ghanem et al., 2013).

Cre is a member of the tyrosine SSR family and is naturally encoded by bacteriophage P1. Cre recombinase is responsible for excising, exchanging or inverting DNA between a pair of 34bp loxP target sites. Each loxP site consists of a core 8bp spacer sequence flanked by two 13bp inverted symmetry regions (half-sites; FIG. 1A). Cre/loxP complex formation begins with site-specific binding of a single Cre molecule to each half-site. Once four Cre molecules bound to two loxP sites come together, the tetrameric synaptic complex is formed and poised for catalysis (Stachowski et al., 2022). DNA recombination takes place at the spacer region in a stepwise manner, involving cleavage and strand exchange, with the ultimate outcome of the recombination reaction determined by the orientation of the spacer sequence (Sternberg et al., 1981; Hoess et al., 1982; Abremski et al., 1983; Lee and Sadowski, 2003).

Initial investigations in the 1980s regarding the role of the spacer sequence introduced the concept that sequence identity between spacers in each of the target substrates is crucial for recombination (Hoess et al., 1986). These studies were corroborated when efficient recombination was shown to occur between a variety of noncanonical spacers providing the spacer sequences are matching (Sheren et al., 2007). The requirement for identical spacer sequences was further explained as a necessity during strand exchange to facilitate effective ligation of the cleaved strand to its complementary strand (Duyne, 2001). Much of our current understanding of lox site preference have been inferred through a series of crystal structures of Cre bound to loxP (Meinke et al., 2016; Duyne 2001, Gopaul et al., 1998; Guo et al., 1997; Grindley, 1997). Protein-DNA interfaces reveal specific residues crucial for half-site recognition, although, direct base contacts between the protein's amino acid side chains and the substrate spacer region are minimal. Nevertheless, the spacer region plays a pivotal role during recombination catalysis, as it directs the order of strand exchange (Meinke et al., 2016; Duyne 2001, Gopaul et al., 1998; Guo et al., 1997; Grindley, 1997).

To enable the direct and site-specific integration at user-defined target sequences, it is necessary to modify DNA recombining enzymes and to change their specificity. So far, DNA recombining enzymes have been evolved on different half-sites of a target site as shown e.g. in WO 2018/229226 and as disclosed in Buchholz and Stewart, 2001. The present invention discloses for the first time that the specificity of a DNA recombining enzyme can be modified based on the spacer sequence of a target site. It is therefore an object of the present invention to provide a spacer-specific DNA recombining enzyme.

### SUMMARY OF THE INVENTION

The objective underlying the present invention is solved by the provision of a method for generating a spacer-specific DNA recombining enzyme (DRE), the method comprising the steps of:
a) providing a 1. library of expression vectors encoding a plurality of 1. variants of a DRE (vDRE), wherein the amino acid sequences of the 1. vDREs comprise one or more amino acid modifications in comparison to a non-variant DRE (nvDRE) from which the 1. vDREs are derived, wherein the nvDRE binds to a first target site comprising in 5′ to 3′ direction a half-site A (HSA), a spacer (Spacer) and a half-site B (HSB), wherein each expression vector comprises:
   (i) a first region comprising a nucleotide sequence encoding one of the 1. vDREs from among the plurality of 1. vDREs operably linked to an expression control sequence, and
   (ii) a second region comprising a nucleotide sequence comprising in 5′ to 3′ direction a first target site, an insert nucleotide sequence (INS) of a length at least 1 nucleotide and a second target site, wherein each of the first and second target site comprises the HSA, a 1. variant spacer (vSpacer) and the HSB, wherein the 1. vSpacer differs by at least one nucleotide from the Spacer;
b) introducing the library of expression vectors into host cells;
c) expressing the plurality of 1. vDREs in the host cells;
d) optionally isolating DNA from the host cells; and
e) determining whether the 1. vDRE shows activity on the target sites of at least one expression vector; optionally repeating steps a) to e) until a 1. vDRE is produced that shows activity on the target sites of at least one expression vector.

According to one embodiment, the method further comprises the step of:
f) providing 1+n. libraries of expression vectors encoding a plurality of 1+n. variants of the vDREs (1+n. vDREs), wherein the 1+n. vDREs comprise one or more amino acid modifications in comparison to the n. vDREs, wherein each expression vector comprises:
   (i) a first region comprising a nucleotide sequence encoding one of the 1+n. vDREs from among the plurality of the 1+n. vDREs operably linked to an expression control sequence, and
   (ii) a second region comprising a nucleotide sequence comprising a first and a second target site 5′ and 3′, respectively, of an insert nucleotide sequence (INS) of a length at least 1 nucleotide, wherein each of the first and second target site comprises the HSA, a 1+n.
      variant spacer (1+n. vSpacer) and the HSB, wherein the 1+n. vSpacer differs by at least one nucleotide from the 1. vSpacer;
g) introducing the 1+n. libraries of expression vectors into host cells;
h) expressing the plurality of 1+n. vDREs in the host cells;
i) isolating DNA from the host cells; and
j) determining whether the 1+n. vDREs show activity on the target sites of at least one expression vector, wherein n≥1.

According to another embodiment, the method further comprising repeating steps f) to j) until a 1+n. vDRE is produced that excises the INS.

According to one embodiment, n is increased by 1 once a 1+n. vDRE is produced that excises the INS.

According to one embodiment, the Spacer and the n or 1+n. variant Spacer each have a length of between 6 and 10 nucleotides, preferably 8 nucleotides. In addition or alternatively, the HSA and the HSB each have a length of between 11 and 15 nucleotides, preferably 13 nucleotides.

According to a further embodiment, the nvDRE is a naturally occurring DRE or a variant thereof that binds to a first and second half site different from the first and second half site bound by the naturally occurring DRE, preferably a tyrosine recombinase or a large serine recombinase,
preferably a tyrosine recombinase selected from the group consisting of Cre-, Dre-, VCre-, SCre-, Vika-, lambda-Int-, F1p-, R-, Kw-, Kd-, B2-, B3-, Nigri- or Panto-recombinases, or
preferably a large serine recombinase selected from the group consisting of A118, TP901, φRV1, Bxb1, φC31, R4, Wβ, Tnpx, Cp36, Dn29, Kp03, Nm60, Pa01, Si74.

According to a further embodiment, step e) or j) of determining whether the 1. vDRE shows activity on the target sites of at least one expression vector comprises:
i) performing PCR on the host cell of c) or h) or the isolated DNA of step d) or i) with a first primer specifically hybridizing 5′ of or partially overlapping or fully overlapping with the first target site, and a second primer specifically hybridizing 3′ of or partially overlapping or fully overlapping with the second target site, and optionally sequencing of the PCR product; or
ii) restriction digestion of the expression vector with one or more restriction enzymes that cleave the INS.

According to yet another embodiment, the method further comprises the step of removing inactive variants of the 1. or 1+n. vDREs from the library of expression vectors.

According to a further aspect, the present invention provides a vDRE obtainable by the method according to any of claims 1 to 8, wherein the amino acid sequence of the vDRE differs in at least one amino acid from the nvDRE. According to a preferred embodiment, the vDRE comprises or consists of an amino acid sequence having at least 85% identity to any one of SEQ ID NOs: 13, 14, or 15.

According to a further aspect, the present invention provides a nucleic acid or group of nucleic acids encoding a vDRE according to the invention.

According to yet another aspect, the present invention provides an expression vector comprising a nucleic acid or group of nucleic acids according to the invention.

According to yet another aspect, the present invention provides a system for (i) integrating a donor nucleic acid into a target nucleic acid, or (ii) exchanging a nucleic acid sequence of interest in the genome of a subject or cell for a different nucleic acid sequence, the system comprising
a polypeptide comprising a vDRE according to the invention, or a nucleic acid or group of nucleic acids according to the invention; and
either (i) a donor nucleic acid to be inserted into the target nucleic acid, or (ii) a nucleic acid sequence differing from the nucleic acid sequence to be exchanged.

According to yet another aspect, the present invention provides a pharmaceutical composition comprising the vDRE according to the invention, the nucleic acid or group of nucleic acids according to the invention, or the expression vector according to the invention, and optionally a pharmaceutically acceptable carrier.

According to a further aspect, the present invention provides the use of the vDRE according to the invention, the nucleic acid or group of nucleic acids according to the invention, the expression vector according to the invention, the system of according to the invention, or the pharmaceutical composition according to the invention, for (i) integrating a nucleic acid sequence of interest into the genome of a subject or cell, or (ii) for exchanging a nucleic acid sequence of interest in the genome of a subject or cell for a different nucleic acid sequence, wherein the cell does not include cells of the human germ line.

According to a further aspect, the present invention provides a vDRE according to the invention, the nucleic acid or group of nucleic acids according to the invention, the expression vector according to the invention, or the pharmaceutical composition according to the invention for use in medicine, preferably for use in the treatment of a genetic disease or disorder, more preferably wherein the genetic disease or disorder is a monogenetic disease or disorder.

According to yet another aspect, the present invention provides a method for treating a genetic disease or disorder in a subject by integrating a donor nucleic acid into a target nucleic acid of the subject, wherein the method comprises the steps of: (i) providing the vDRE according to the present invention, the nucleic acid or group of nucleic acids of the present invention, the expression vector of the present invention, the pharmaceutical composition of the present invention, or the system of the present invention, in a therapeutically effective amount, and a donor nucleic acid, optionally allowing the vDRE to be expressed, and (ii) allowing the vDRE to integrate the donor nucleic acid into the target nucleic acid.

According to yet another aspect, the present invention provides a method for treating a genetic disease or disorder in a subject by exchanging a nucleic acid present in the genome of a subject or a cell, wherein the method comprises the steps of: (i) providing the vDRE according to the present invention, the nucleic acid or group of nucleic acids of the present invention, the expression vector of the present invention, the pharmaceutical composition of the present invention, or the system of the present invention, and a different nucleic acid that shall replace the nucleic acid in the genome of the subject or the cell, optionally allowing the vDRE to be expressed, and (ii) allowing the vDRE to exchange the nucleic acid in the genome of the subject or cell and insert the different nucleic acid.

Further aspects and embodiments of the invention will become apparent from the appending claims and the following detailed description.

### DESCRIPTION OF THE DRAWINGS

The invention is further illustrated by the following figures and examples without being limited thereto.

FIG. 1A shows the loxP sequence with top (TS) and bottom strand (BS). Bases in the spacer region are labelled and highlighted in teal. The respective attack points of the catalytic tyrosine (Y324) to the DNA strands are indicated by triangles. FIG. 1B is a plasmid map schematic showing the assembled pGG and target library plasmid for the recombinase activity assay. The amplified spacer library, with lox sites labeled and depicted as two rectangles, is assembled to the E. coli recombinase expression vector (pGG) using Golden Gate assembly at BbsI restriction sites. FIG. 1C shows the pEVO plasmid used for site-directed evolution of the inserted recombinase. Both vectors (FIG. 1B, FIG. 1C) contain a Chloramphenicol resistance gene (CmR) for selection and an L-arabinose (labeled araC) inducible promoter (labeled pBAD promoter) system for controlling recombinase expression. The DNA sequences encoding the recombinase are inserted to the pGG with BsrGI and SbfI restriction sites and BsrGI/XbaI restriction sites for the pEVO. active recombinases selected with restriction digest of AvrII/NdeI. FIG. 1D illustrates in both, the top and the bottom panel the plasmid-based recombinase activity assay. To visually compare the non-recombined and recombined plasmids in a sample, the purified plasmid DNA from an E. coli overnight culture is digested using BsrGI and XbaI (for assaying pEVO, top panel) or using BsrGI and PacI (for assaying pGG, bottom panel). The digest linearizes the DNA to easily compare the smaller recombined plasmids to the non-recombined plasmids in the sample with gel electrophoresis.

FIG. 2 is a schematic overview of the target site screen. i) Construction of target library around the spacer sequence of the loxP site. Examples of the matching spacers are shown, with alterations to the canonical loxP spacer sequence highlighted in light grey. ii) Library cloning into an E. coli recombinase expression vector. iii) Assembled library vector transformation to E. coli and induced to express Cre. The editing outcomes are indicated by the teal target sites where an active event is recombined and an inactive event is non-recombined. iv) Purification of vectors and subsequent amplification over the target sites with primers containing Illumina indexing sequence. v) Quantification of the recombination rate for each target site calculated from deep sequencing reads (%).

FIG. 3 shows the target site screen results plotted as activity ratio of Cre recombination (fraction of recombination) for each target site. Activity ratio is calculated for each target site by dividing the activity by the highest activity in the screen. Each dot (teal) represents a unique spacer sequence within the library. Dotted vertical lines outline the range where the targets are efficiently recombined by Cre, defined by recombination rate±25% loxP. Plot zoom showing targets with lowest activity. Targets selected for evolution are labeled loxSE1, loxSE2 and loxSE3.

FIG. 4A shows the impact of number of mismatches in the spacer to Cre recombination efficiency. Cre recombination is represented as activity ratio and effects for one (n=36), two (n=238), three (n=831), four (n=1712), five (n=1999), six (n=988), seven (n=128) and eight (n=12) mismatches. The wild-type loxP is plotted as a line at 0 mismatches. FIG. 4B and FIG. 4C show sequence logos representing spacer preference of Cre. The base height in each logo is calculated by relative frequency in the given subpopulation. The base frequency is normalized to library representation. The logo in FIG. 4B is calculated from a subpopulation of spacer sequences with the highest recombination efficiency or top 10% recombined spacers (n=595 targets). The logo in FIG. 4C is calculated from a subpopulation of spacer sequences with the lowest recombination efficiency or bottom 10% recombined spacers (n=595 targets). FIG. 4D shows a spacer specificity profile of Cre generated from the recombination rates of all target library variants. The heatmap grey-scale, and corresponding fold change in each tile, represent the effect of the base change at each position on Cre recombination relative to Cre/loxP recombination. The fold change is calculated from the binomial GLM coefficients. The canonical loxP bases are outlined in black for each base position in the spacer. FIG. 5A shows the target site sequences used for evolution aligned to loxP. The spacer base sequence of loxP is indicated in bold letters. Base mismatches from loxP are displayed in light grey for the loxSE1, loxSE2 and loxSE3 spacer sequences (written in 5′→3′ direction). FIG. 5B schematically illustrates the evolution process. i) Recombinase library assembly to evolution vector. ii) Transformation of assembled vectors to E. coli for induction of recombinase expression. iii) Vector purification and selection for active recombinase variants by linearization of non-recombined vectors carrying the inactive recombinase variant. iv) Error-prone amplification with primers p5 and p6 to insert new mutations to the active recombinase library. Only the non-linearized recombined vectors result in an amplification product and are carried on to the next cycle of evolution. FIG. 5C shows the activities of the plasmid-based restriction assays for Cre, LSE1, LSE2 and LSE3 starting libraries (cycle 1) and ending libraries (cycle 11) on target sites loxP, loxSE1, loxSE2 and loxSE3. Two triangles indicate the non-recombined substrate (5 kb), and one triangle indicates the recombined substrate (4.2 kb).

FIG. 6A illustrates violin plots showing the distribution of recombination (%) (y-axis) for each recombinase variant in the final evolved libraries (after 11 rounds of evolution) and Cre activity on target sites loxP, loxSE1, loxSE2 and loxSE3 (x-axis). The width of each curve in the violin plot corresponds to the approximate frequency of recombinase variants with the equivalent recombination (%). FIG. 6B illustrates scatterplots showing the recombination (%) of each library of their evolved target site along the y-axis compared to recombination (%) of loxP along the x-axis. Each dot represents a unique recombinase and the grey-scale indicates the library of the recombinase. The upper left grey square in each plot highlights recombinases specific for the evolved site. The recombinases that were further analyzed are labeled: RecS1, RecS2 and RecS3. FIG. 6C illustrates the plasmid-based restriction assays showing Cre, RecS1, RecS2 and RecS3 activity for loxP and the evolved target sites loxSE1, loxSE2 and loxSE3. M=GeneRuler DNA Ladder Mix 10kb. The bar plots show the mean recombination (%) of the activities quantified from the band intensities of the plasmid-based restriction assay. The restriction assays were done in triplicates (n=3). The points represent each individual replicate recombination (%).

FIGS. 7A-7D illustrate the target site library activity screen of RecS3. FIG. 7A shows a comparison of Cre spacer library activity (x-axis) and RecS3 spacer library activity (y-axis). Each dot represents a unique spacer in the library. The activity ratio for each recombinase was calculated by dividing the activity by the highest activity in the respective screen. Activity ratio is plotted on a square root scale for both the x-and y-axis. FIG. 7B and FIG. 7C show sequence logos depicting spacer preference of RecS3. The base height in each logo is calculated by relative frequency in the given subpopulation. The base frequency is normalized to library representation. The logo in FIG. 7B is calculated from a subpopulation of spacer sequences with the highest recombination efficiency or top 10% recombined spacers (n=595 targets). The logo in FIG. 7C is calculated from a subpopulation of spacer sequences with the lowest recombination efficiency or bottom 10% recombined spacers (n=595 targets). The loxSE3 bases are shown in dark grey. FIG. 7D shows the spacer specificity profile of RecS3 generated from the recombination rates of all spacer library variants. The heatmap grey-scale, and corresponding fold change in each tile, represent the effect of the base change at each position on RecS3 recombination relative to RecS3/loxSE3 recombination. The fold change is calculated from the binomial GLM coefficients. The loxSE3 bases are outlined in black for each base position in the spacer.

FIGS. 8A-8B illustrate the restriction assay of RecS3 and indicated RecS3 variants. LoxP and loxSE3 activity of Cre^{wt}, RecS3, and eight RecS3 mutants with single residue mutated back to Cre^{wt} are shown. Restriction assay displays amount of non-recombined plasmid (indicated by two triangles) and recombined plasmids (indicate by one triangle) in the sample. Bottom graphs plot the correlated mean recombination (%) of the above gel image calculated from gel band intensities. RecS3 recombination in teal and Cre recombination in dark gray. Bar represents mean of n=3 replicates. P values are reported from unpaired two-tailed t-test ns: p>0.05, *: p<0.05, **: p <0.01, * * * : p<0.001, ****: p<0.0001.

FIGS. 9A-9B show the mutational analysis of Cre with RecS3 mutations (analogous to FIGS. 8A-8B). P values are reported from unpaired two-tailed t-test ns: p>0.05, *: p<0.05, **: p<0.01, * * * : p<0.001, ****: p<0.0001.

FIG. 10A shows a summary of MD-based analysis of loxP and loxSE3 recognition by wild-type Cre and mutant variants. The plot shows interactions observed in the last 100 ns of MD simulations between the DNA and Y324 in the active (A) and inactive (I) monomers. DNA numbering according to FIG. 5A. FIG. 10B shows details of the MD-refined structure of the RecS3/loxP complex. FIG. 10C shows details of the MD-refined structure of the RecS3S320I/loxP complex. Active and inactive recombinase monomers are shown in pale and blue cartoons, respectively. Side chains of residues Y324 and 320 and DNA bases are labeled and shown in atom-colored sticks. The surface mesh denotes van der Waals contacts between protein residues at position 320 and DNA bases T3′(TS) and G4(BS). Black dashed indicate H-bonds.

FIG. 11 is a cartoon putty representation of RecS3 in complex with loxP. RecS3 is shown in grey-scale and rendered in accordance to RMSDCα values relative to Cre (as indicated by the gradient side bar). Active (A) and inactive (I) RecS3 monomers, respectively, are shown, and helix M(I) is highlighted with a square. LoxP is shown in grey ladder representation.

### LIST OF SEQUENCES

The sequences referred to herein are disclosed in detail in the accompanying sequence listing. Exemplary sequences of the present invention are also listed in the following table.

| SEQ | | |
|---|---|---|
| ID | | Sequence (amino acid (N→C) |
| NO: | Name | or nucleic acid (5′→3′)) |
| 1 | BbsI | ACACCGGGTCTTC |
| | restriction | |
| | site | |
| | downstream | |
| 2 | BbsI | GAAGACCTGTTTA |
| | restriction | |
| | site upstream | |
| 3 | fully | AAAATTTT |
| | symmetric | |
| | spacer | |
| 4 | loxSE1 | ATAACTTCGTATAACGTGATCTATACGAAG |
| | | TTAT |
| 5 | loxSE2 | ATAACTTCGTATACAGTATAATATACGAAG |
| | | TTAT |
| 6 | loxSE3 | ATAACTTCGTATAAACCGGTCTATACGAAG |
| | | TTAT |
| 7 | loxP | ATAACTTCGTATAATGTATGCTATACGAAG |
| | | TTAT |
| 8 | Cre (wt) | MSNLLTVHQNLPALPVDATSDEVRKNLMDM |
| | | FRDRQAFSEHTWKMLLSVCRSWAAWCKLNN |
| | | RKWFPAEPEDVRDYLLYLQARGLAVKTIQQ |
| | | HLGQLNMLHRRSGLPRPSDSNAVSLVMRRI |
| | | RKENVDAGERAKQALAFERTDFDQVRSLME |
| | | NSDRCQDIRNLAFLGIAYNTLLRIAEIARI |
| | | RVKDISRTDGGRMLIHIGRTKTLVSTAGVE |
| | | KALSLGVTKLVERWISVSGVADDPNNYLFC |
| | | RVRKNGVAAPSATSQLSTRALEGIFEATHR |
| | | LIYGAKDDSGQRYLAWSGHSARVGAARDMA |
| | | RAGVSIPEIMQAGGWTNVNIVMNYIRNLDS |
| | | ETGAMVRLLEDGD |
| 9 | spacer variant | CAGTATTC |
| 10 | loxP spacer | ATGTATGC |
| 11 | spacer variant | AATGTGTC |
| 12 | spacer variant | TGAATTCG |
| 13 | RecS1 | MSNLLTIHQNLPALPVDATSDEVRKNLMDM |
| | | FRDRQAFSEHTWKMLLSVCRSWAAWCRLNN |
| | | RKWFPAEPEDVRDYLLYLQARGLAVKTIQQ |
| | | HLGQLNMLHRRSGLPQPSDSNAVSLVMRRI |
| | | RKENVDAGERAIQALAFERTDFDQVRSLME |
| | | NSDRCQDIRNLAFLGIAYNTLLRIAEIARI |
| | | RVKDISRTDGGRMLIHIGRTKTLVSTAGVE |
| | | KALSLGVTKLVERWISVSGVADDPNNYLFC |
| | | RVRKNGVAAPSATSQLSTRALEGIFEATHR |
| | | LIYGAKDDSGQRYLAWSGHSARVGAARDMA |
| | | RAGVSIPEIMQTGGWTNVNIVMNYIRNLDS |
| | | ETGAMVRLLEDGD |
| 14 | RecS2 | MSNLLTVHKNLPALPVDATSDEVRKNLTDM |
| | | FRDRQAFSEHTWEALLSVCRSWAAWCELNN |
| | | RKWFPAEPEDVRDYLLHLQARGLAVNTVQQ |
| | | HLARLNMLHRRFGLPRPSDSNAVSLVMRRI |
| | | RKENVDAGERAKQALAFERTDFDQVRSLME |
| | | NSDRCQDIRNLAFLGIAYNTLLRIAEIARV |
| | | RVKDISRTDGGRMLIHIGRTKTLVSTAGVE |
| | | KALSLGVTKLVERWISVSGVADDPNNYLFC |
| | | RVRKNGVAAPSATSQLSTRALEGIFEATHR |
| | | LIYGAKDDSGQRYLAWSGHSARVGAARDMA |
| | | RAGVSIPEIMQAGGWTDVNIVMNYIRNLDS |
| | | ETGAMVRLLEDDD |
| 15 | RecS3 | MSNLPTAHQNLPALPVDATSDEVRKNLMDM |
| | | FRDRQAFSEHTWKMLLSVCRSWAAWCKLNN |
| | | RKWFPAEPEDVRDYLLYLQARGLAVKTIQQ |
| | | HLGQLNMLHRRSGLPRPSDSNAVSLVMRRI |
| | | RKENVDAGERAEQALAFERTDFDQVRSLME |
| | | NSDRCQDIRNLAFLGIAYNTLLRIAEIARI |
| | | RVKDISRTDGGRMLIHIGRTKTLVSTAGVE |
| | | KALSLGVTKLVERWISVSGVADDPNNYLFC |
| | | RVRKNDVAAPSATSQLSTRALEGIFEATHR |
| | | LIYGAKDDSGQRYLAWSGHSARVGAARDMA |
| | | RAGVSIPEIMQAGGWITVNSVMYYIRNLDS |
| | | ETGAMVRLLEDGD |
| 16 | loxSE1 spacer | ACGTGATC |
| 17 | loxSE2 spacer | CAGTATAA |
| 18 | loxSE3 spacer | AACCGGTC |
| 19 | loxP variant | ATAACTTCGTATAAGGTATGTTATACGAAG |
| | | TTAT |
| 20 | loxP variant | ATAACTTCGTATAAGGTATGCTATACGAAG |
| | | TTAT |
| 21 | loxP variant | ATAACTTCGTATAAGATATGCTATACGAAG |
| | | TTAT |
| 22 | loxP variant | ATAACTTCGTATAAGGTACTCTATACGAAG |
| | | TTAT |
| 23 | spacer variant | AGGTATGC |
| 24 | spacer variant | ATGTATTC |
| 25 | spacer variant | ATGTATGA |
| 26 | Primer P1 | TTTGAAGACCCCACCCGCAAGCTTCACGG |
| 27 | Primer P2 | TTTGAAGACCTAAACGCACCGAGCACGC |
| 28 | Primer P3 | GTGACTGGAGTTCAGACGTGTGCTCTTCCG |
| | | ATCTGTATGACATGTCGCGAAACACC |
| 29 | Primer P4 | ACACTCTTTCCCTACACGACGCTCTTCCGA |
| | | TCTCTATGCATAAACGCACCGAGC |
| 30 | Primer P5 | GCGTCACACTTTGCTATGCC |
| 31 | Primer P6 | GTTCGCCAGTTAATAGTTTGCTGC |

### DETAILED DESCRIPTION OF THE INVENTION

Before the present invention is described in detail below, it is to be understood that this invention is not limited to the particular methodology, protocols and reagents described herein as these may vary.

It is also to be understood that the terminology used herein is for the purpose of describing particular embodiments only, and it is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Klbl, H. eds. (1995), Helvetica Chimica Acta, CH-4010 Base1, Switzerland).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. In the following passages, different aspects of the invention are defined in more detail. Each aspect so defined may be combined with any other aspect or aspects unless clearly indicated to the contrary. Any feature indicated as being optional, preferred or advantageous may be combined with any other feature or features indicated as being optional, preferred or advantageous.

Several documents are cited throughout the text of this specification. Each of the documents cited herein (including all patents, patent applications, scientific publications, manufacturer's specifications, instructions etc.), whether supra or infra, is hereby incorporated by reference in its entirety. Nothing herein is to be construed as an admission that the invention is not entitled to antedate such disclosure by virtue of prior invention. Some of the documents cited herein are characterized as being “incorporated by reference”. In the event of a conflict between the definitions or teachings of such incorporated references and definitions or teachings recited in the present specification, the text of the present specification takes precedence.

In the following, the elements of the present invention will be described. These elements are listed with specific embodiments; however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

### Definitions

In the following, some definitions of terms frequently used in this specification are provided. These terms will, in each instance of its use, in the remainder of the specification have the respectively defined meaning and preferred meanings.

As used in this specification and the appended claims, the singular forms “a”, “an”, and “the” include plural referents, unless the content clearly dictates otherwise.

The “percentage of sequence identity” is determined by comparing two optimally aligned sequences over a comparison window, wherein the portion of the sequence in the comparison window can comprise additions or deletions (i.e. gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “identical” is used herein in the context of two or more nucleic acids or polypeptide sequences, to refer to two or more sequences or subsequences that are the same, i.e. that comprise the same sequence of nucleotides or amino acids. Sequences are “identical” to each other if they have a specified percentage of nucleotides or amino acid residues that are the same. According to the present invention, at least 85% identical includes at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.2%, at least 99.5%, at least 99.6%, at least 99.7%, at least 99.8%, at least 99.9% or 100% identity over the specified sequence, when compared and aligned for maximum correspondence over a comparison window, or designated region as measured using one of the following sequence comparison algorithms or by manual alignment and visual inspection. These definitions also refer to the complement of a test sequence. The term “at least 85% sequence identity” is used throughout the specification with regard to polypeptide and polynucleotide sequence comparisons. This expression preferably refers to a sequence identity of at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.2%, at least 99.5%, at least 99.6%, at least 99.7%, at least 99.8%, at least 99.9%, or 100% to the respective reference polypeptide or to the respective reference polynucleotide.

The term “sequence comparison” is used herein to refer to the process wherein one sequence acts as a reference sequence, to which test sequences are compared. When using a sequence comparison algorithm, test and reference sequences are entered into a computer, if necessary, subsequence coordinates are designated, and sequence algorithm program parameters are designated. Default program parameters are commonly used, or alternative parameters can be designated. The sequence comparison algorithm then calculates the percent sequence identities for the test sequences relative to the reference sequence, based on the program parameters. In case where two sequences are compared and the reference sequence is not specified in comparison to which the sequence identity percentage is to be calculated, the sequence identity is to be calculated with reference to the longer of the two sequences to be compared, if not specifically indicated otherwise. If the reference sequence is indicated, the sequence identity is determined on the basis of the full length of the reference sequence indicated by one of the SEQ ID NOs of the present invention, if not specifically indicated otherwise.

The term “nucleic acid” and “nucleic acid molecule” are used synonymously herein and are understood as well-accepted in the art, i.e. as single or double-stranded oligo- or polymers of deoxyribonucleotide or ribonucleotide bases or both. The term “nucleic acids” as used herein includes not only deoxyribonucleic acids (DNA) and ribonucleic acids (RNA), but also all other linear polymers in which the bases adenine (A), cytosine (C), guanine (G) and thymine (T) or uracil (U) are arranged in a corresponding sequence (nucleic acid sequence). The invention also comprises the corresponding RNA sequences (in which thymine is replaced by uracil), complementary sequences and sequences with modified nucleic acid backbone or 3′ or 5′-terminus. Nucleic acids in the form of DNA are however preferred.

The term “DNA recombining enzyme”, abbreviated as DRE, as used herein refer to an enzyme that is capable of manipulating the structure of a genome by integrating a nucleic acid into the genome of a subject or a cell. A DRE is preferably present in form of a monomer, a dimer or a tetramer, in particular in form of a dimer. Two dimers usually work together to catalyze a recombination reaction. Dimers and tetramers can comprise two or four identical protein monomers (homodimer or homotetramer), respectively, or alternatively two or more different monomers (heterodimer or heterotetramer). DREs according to the present invention include but are not limited to site-specific recombinases such as a serine and tyrosine site-specific recombinases, and large serine recombinases (also called integrases). In accordance with one preferred embodiment of the present invention, the DNA recombining enzyme is preferably a recombinase, more preferably a tyrosine recombinase, and most preferably a tyrosine recombinase selected from the group consisting of Cre and Cre-derived recombinases, Vika (disclosed e.g. in EP 2690177 A1 and incorporated herein by reference in its entirety), Panto (disclosed e.g. in EP 3263708 A1 and incorporated herein by reference in its entirety), Dre, D7L, D7R, Nigri (disclosed e.g. in EP 2877585 A1 and incorporated herein by reference in its entirety), VCre, SCre, YR1, YR2, YR4, YR6, YR8, YR9, YR11, YR12 (Jelicic et al. 2023), Tre, Brec1 and recombinases derived therefrom. According to a further preferred embodiment, the DNA recombining enzyme is an engineered site-specific variant of a naturally occurring DNA recombinase. According to a further preferred embodiment, the DNA recombining enzyme is selected from the group consisting of A118, TP901, φRV1 (also termed PhiRv1), φC31, R4, Wβ, Tnpx, Cp36, Dn29, Kp03, Nm60, Pa01, Si74 lambda-Int, F1p, Kd, Kw, B2, and B3, piggyBac, sleeping beauty, topoisomerases, Bxb1 and enzymes derived therefrom. According to a further preferred embodiment, the DNA recombining enzyme is selected from the group consisting of Bxb1, PhiC31, Sh25, Si74, Bm99, Me99, Ma37, Nm60, Cc91, Vh19, Cs56, Bt24, No67, Fm04, Bu30, Ma05, Rh64, Cb16, uCb4, Ec03, Ec04, Ec05, Ec06, Ec07, Ef01, Ef02, Kp01, Kp03, Kp04, Kp05, Pa01, Pa03, Sa01, Sa02, Pf13, Td08, Se37,

Ct03, Cd31, Ps40, Sa10, Td01, Enc3, Fp10, Ph43, Sm18, Cd16, Pf80, Bs46, Pf48, Rb27, Sa51, Bc30, Cd04, Cd15, Sa34, Pp20, R109, Efs2, Pf15, Ps45, Sp56, Dn29, Vh73, Em12, Pc64, Vp82, Cp36, Pc01, Enc9 (Durrant et al., 2023). According to a particularly preferred embodiment, the DNA recombining enzyme is a site-specific recombinase or a large serine recombinase (integrase).

The terms “DRE variant(s)” and “variant(s) of a DRE” are used interchangeably herein and denote a DRE differing from a non-variant DRE in at least one amino acid, for example an amino acid substitution, deletion or addition, preferably an amino acid substitution.

The term “engineered DNA recombining enzyme” as used herein refers to any naturally occurring DNA recombining enzyme that has been further modified, e.g. evolved as described herein, in particular to modify its target-site specificity and/or its activity and/or efficiency.

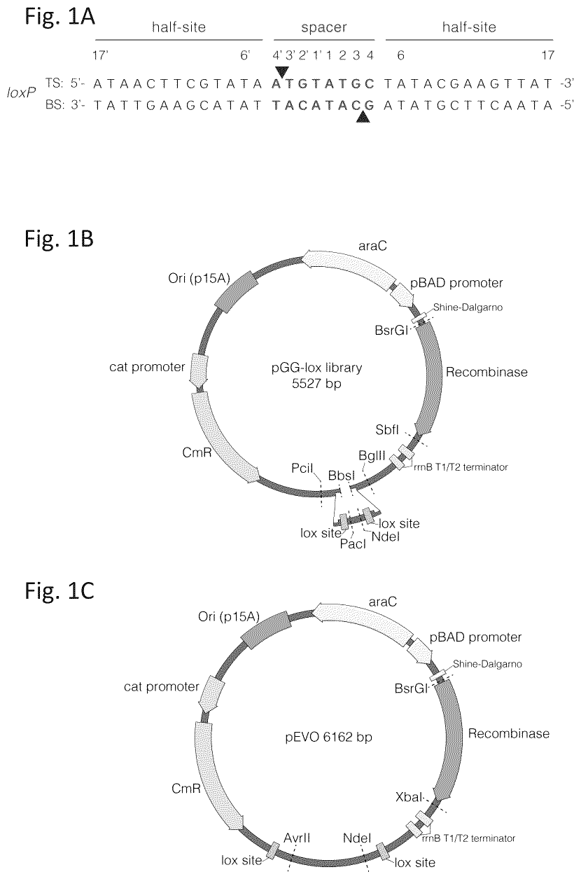

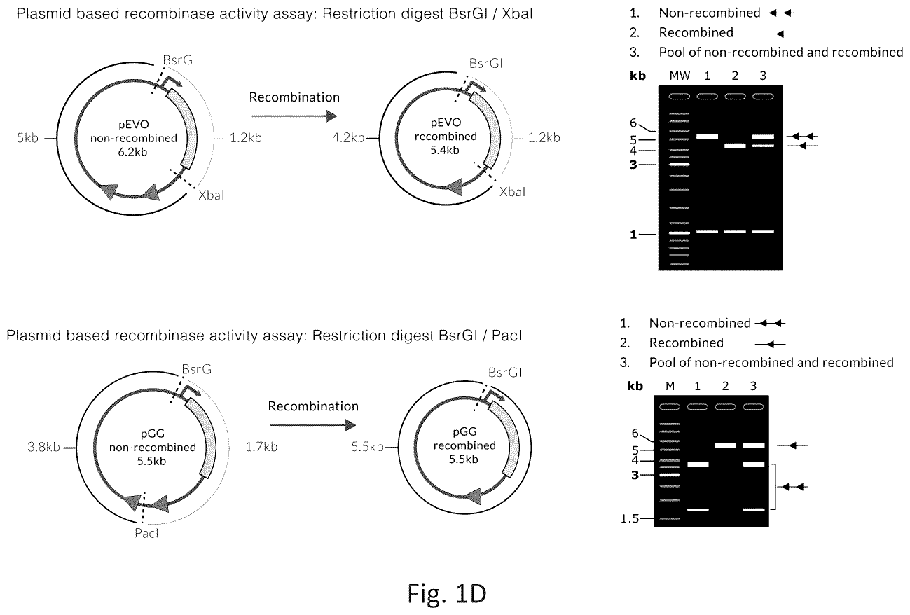

The term “target site” (also referred to as “recognition site” or “target sequence”) as used herein refers to a specific nucleotide sequence which a DNA recombining enzyme recognizes. In cases of recombinases being the DNA recombining enzyme, for example, the target site is the site at which DNA breakage and strand exchange occur. Such target sequences typically range between 30 and 200 base pairs in length and are comprised of two inversely repeated recombinase binding regions (first and second half-site) flanking a central spacer sequence (Meinke et al., 2016). An example of such a recognition site can be seen in the SSR Cre/loxP binding complex, where the Cre recombinase is bound to the 34 base pair loxP target sequence. The loxP recognition site comprises two 13 base pair inverted repeat Cre binding elements flanking an 8 base pair spacer region. The left half-site (also referred to as half-site A) is the 13 base pair binding element to the left of the spacer and the right half-site (also referred to as half-site B) is the 13 base pair binding element to the right of the spacer seen in 5′ to 3′ direction (FIG. 1A). Depending on the number and relative orientation of the recognition sites and their spacers, the DNA recombining enzyme either performs an excision, an integration, an inversion or a replacement of genetic content (reviewed in Meinke et al., 2016). Therefore, according to a preferred embodiment, a “target site” is a nucleotide sequence comprising a first half-site, a second half-site, and a spacer separating the first and the second half-site. For a recombination event to occur, a recombinase enzyme complex recognizes a first target site and a second target site on a DNA double strand. The target sites are also referred to as upstream and downstream target sites, depending on their location on the DNA double strand. In symmetric target sites, the first half-site (e.g. the left half-site) and the second half-site (e.g. the right half-site) are identical and palindromic (reverse complement). In asymmetric target sites, the first half-site (e.g. the left half-site) and the second half-site (e.g. the right half-site) are not identical and not palindromic, i.e. they differ from each other in at least one nucleotide.

The term “therapeutically effective amount” as used herein, means that amount of active compound or pharmaceutical agent that elicits the biological or medicinal response in a cell, tissue system, animal or human being sought by a researcher, veterinarian, medical doctor or other clinician, which includes alleviation of the symptoms of the disease or disorder being treated.

The term “pharmaceutical composition” as used herein refers to a substance and/or a combination of substances being used for the identification, prevention or treatment of a disease or tissue status. The pharmaceutical composition is formulated to be suitable for administration to a patient in order to prevent and/or treat a disease. Further, a pharmaceutical composition refers to the combination of an active agent with a carrier, inert or active, making the composition suitable for therapeutic use. Such a carrier is also referred to as being pharmaceutically acceptable. Pharmaceutical compositions can be formulated for oral, parenteral, topical, inhalative, rectal, sublingual, transdermal, subcutaneous or vaginal application routes according to their chemical and physical properties. Pharmaceutical compositions comprise solid, semisolid, liquid, transdermal therapeutic systems (TTS). Solid compositions are selected from the group consisting of tablets, coated tablets, powder, granulate, pellets, capsules, effervescent tablets or transdermal therapeutic systems. Also comprised are liquid compositions, selected from the group consisting of solutions, syrups, infusions, extracts, solutions for intravenous application, solutions for infusion or solutions of the carrier systems of the present invention. Semisolid compositions that can be used in the context of the invention comprise emulsion, suspension, creams, lotions, gels, globules, buccal tablets and suppositories.

As used herein, the term “pharmaceutically acceptable” embraces both, human and veterinary use. For example, the term “pharmaceutically acceptable” embraces a veterinary acceptable compound or a compound acceptable in human medicine and health care.

The term “subject” as used herein, refers to an animal, preferably a mammal, most preferably a human.

The term “plurality” as used herein includes any number of events described by an integer above one. The term “more than one” can be substituted for the term “plurality”.

### DESCRIPTION OF EMBODIMENTS

The present invention is in the field of DNA recombining enzymes (DREs). In order to study the role of spacer sequences during recombinase-mediated recombination, a method to comprehensively quantify recombination efficiencies across a large number of predefined target sequences was developed. This method was applied to a library of about 6,000 different loxP-like sites, where identity was maintained between the two spacers. Cre recombinase demonstrated efficient recombination with 84% of the spacer sequences within the target site library. To assess the feasibility of reprogramming recombinase spacer specificity, targets with spacer sequences inefficiently recombined were selected to evolve recombinase libraries for increased activity. Analysis of recombinase variants from these libraries showed that selectivity of the spacer sequence can successfully be altered. The present invention highlights the ability to leverage spacer specificity to enhance the recombination properties of tailor-made DRE systems.

The present invention provides the first method for changing the specificity of a DRE on the basis of the spacer sequence. Specifically, the present invention provides a method for generating a spacer-specific DNA recombining enzyme (DRE), the method comprising the steps of:
a) providing a 1. library of expression vectors encoding a plurality of 1. variants of a DRE (vDRE), wherein the amino acid sequences of the 1. vDREs comprise one or more amino acid modifications in comparison to a non-variant DRE (nvDRE) from which the 1. vDREs are derived, wherein the nvDRE binds to a first target site comprising in 5′ to 3′ direction a half-site A (HSA), a spacer (Spacer) and a half-site B (HSB), wherein each expression vector comprises:
   (i) a first region comprising a nucleotide sequence encoding one of the 1. vDREs from among the plurality of 1. vDREs operably linked to an expression control sequence, and
   (ii) a second region comprising a nucleotide sequence comprising in 5′ to 3′ direction a first target site, an insert nucleotide sequence (INS) of a length at least 1 nucleotide and a second target site, wherein each of the first and second target site comprises the HSA, a 1. variant spacer (vSpacer or 1. vSpacer) and the HSB, wherein the 1. vSpacer differs by at least one nucleotide from the Spacer;
b) introducing the library of expression vectors into host cells;
c) expressing the plurality of 1. vDREs in the host cells;
d) optionally isolating DNA from the host cells; and
e) determining whether the 1. vDRE shows activity on the target sites of at least one expression vector. The method optionally further comprises repeating steps a) to e) until a 1. vDRE is produced that shows activity on the target sites of at least one expression vector.

In accordance with the present invention, a variant—be it the first (1.), second, third or any subsequent variant (1+n. variant)—of a DRE differs from a known, initial, starting or previous DRE in at least one amino acid. That is, one or more mutations are introduced into the sequence encoding the DRE, which leads to at least one or more amino acid substitution, deletion or addition, respectively, preferably to at least one or more amino acid substitution. Thus, according to a particularly preferred embodiment, the variant DRE differs from the previous DRE (be it the known, initial or starting DRE such as the nvDRE, or a preceding variant DRE) in at least one, two, three, four, five, six, seven, eight, nine, ten, or more amino acids, preferably in at least 15, 20, 25, 30, 35, 40, 45, 50, 55, 60, 65, 70, 75, 80, 85, 90, 95, or 100 amino acids. Most preferably, the variant DRE differs from the previous DRE in between 1 and 10 amino acids. For evolving DREs, the first variant to be generated in accordance with a preferred embodiment of the present invention differs from a known DRE in at least one, two, three, four, five, six, seven, eight, nine, or ten or more amino acids, preferably in at least 15, 20, 25, 30, 35, 40, 45, 50, 55, 60, 65, 70, 75, 80, 85, 90, 95, or 100 amino acids. Any subsequent vDRE (also referred to as 1+n. variant) preferably differs from the initially generated or previous vDRE in at least one, two, three, four, five, six, seven, eight, nine, ten, or more amino acids, preferably in at least 15, 20, 25, 30, 35, 40, 45, 50, 55, 60, 65, 70, 75, 80, 85, 90, 95, or 100 amino acids. The same applies to each following vDRE, which preferably differs from the previous vDRE in at least one, two, three, four, five, six, seven, eight, nine, ten, or more amino acids, preferably in at least 15, 20, 25, 30, 35, 40, 45, 50, 55, 60, 65, 70, 75, 80, 85, 90, 95, or 100 amino acids. It is to be understood that the term “non-variant DRE” or “nvDRE” denotes the starting DRE, the term “first DRE variant” or “1. variant(s) of a DRE” or “1. vDRE” denotes the first variant of the DRE derived from the nvDRE, and the term “further variant(s) of a DRE” or “1+n. vDRE” denotes each following DRE. In other words, the term “1+n. vDRE” denotes the variant DRE derived from the first DRE variant, and depending on the integer chosen for n, a respective following variant DRE, such as e.g. the second variant DRE if n=1 derived from the first variant DRE, the third variant DRE if n=2 derived from the second variant DRE, the fourth variant DRE if n=3 derived from the third variant DRE and so on.

According to the present invention, n is an integer, preferably an integer selected between 1 and 50, more preferably between 1 and 20. The term “1+n.” as used herein generally refers to a number of variants generated in addition to the first variant, denoted as 1. variant. With n being an integer, the number of variants increases in addition to the first variant depending on the choice of n.

Variants of a DRE can be generated using any conventional method known in the art. According to a preferred embodiment, the variants are generated by amplifying the respective DRE gene with error-prone PCR to create a library of variants. According to a particularly preferred embodiment of the present invention, a low-fidelity DNA polymerase is used for error-prone PCR.

According to one particularly preferred embodiment, the DRE variants are based on a naturally occurring DRE. According to one such embodiment, the naturally occurring DRE is selected from the group consisting of but not limited to Cre and Cre-derived recombinases, Vika, Panto, Dre, D7L, D7R, Nigri, VCre, SCre, YR1, YR2, YR4, YR6, YR8, YR9, YR11, YR12, Tre, Brec1, A118, TP901, φRV1 (also termed PhiRv1), φC31, R4, Wβ, Tnpx, Cp36, Dn29, Kp03, Nm60, Pa01, Si74 lambda-Int, F1p, Kd, Kw, B2, B3, piggyBac, sleeping beauty, topoisomerases, Bxb1, PhiC31, Sh25, Si74, Bm99, Me99, Ma37, Nm60, Cc91, Vh19, Cs56, Bt24, No67, Fm04, Bu30, Ma05, Rh64, Cb16, uCb4, Ec03, Ec04, Ec05, Ec06, Ec07, Ef01, Ef02, Kp01, Kp03, Kp04, Kp05, Pa01, Pa03, Sa01, Sa02, Pf13, Td08, Se37, Ct03, Cd31, Ps40, Sa10, Td01, Enc3, Fp10, Ph43, Sm18, Cd16, Pf80, Bs46, Pf48, Rb27, Sa51, Bc30, Cd04, Cd15, Sa34, Pp20, R109, Efs2, Pf15, Ps45, Sp56, Dn29, Vh73, Em12, Pc64, Vp82, Cp36, Pc01, and Enc9. Particularly preferred are tyrosine and large serine recombinases. More preferred are tyrosine recombinases selected from the group consisting of Cre-, Dre-, VCre-, SCre-, Vika-, lambda-Int-, F1p-, R-, Kw-, Kd-, B2-, B3-, Nigri- or Panto-recombinases, or large serine recombinases selected from the group consisting of A118, TP901, φRV1, Bxb1, φC31, R4, Wβ, Tnpx, Cp36, Dn29, Kp03, Nm60, Pa01, Si74.

Thus, and in accordance with the present invention, for generating DRE variants based on a naturally occurring DRE, the nucleic acid sequence encoding said naturally occurring DRE, for example the coding sequence of Cre, is subject to the method of the invention by introducing one or more mutations into the coding sequence thereof, e.g. by error-prone PCR as described herein. For generating

DRE variants based on a non-naturally occurring DRE, the nucleic acid sequence encoding said non-naturally occurring DRE is subject to the method of the invention by introducing one or more mutations into the coding sequence thereof, e.g. by error-prone PCR as described herein. Thus, according to an alternatively particularly preferred embodiment, the DRE variants are based on a non-naturally occurring DRE, such as a naturally-occurring DRE that has already been modified, or such as an artificially created DRE, also called designer DRE.

The first target site is the target site of the respective nvDRE on the basis of which the 1. variants of the DRE are generated. For example, in cases of a naturally occurring DRE to be the starting point of the method of the present invention and thus the template for the mutations in step a) leading to the 1. variants of the DRE, the first target site is the one of the naturally occurring DRE. An example of such a naturally occurring DRE is Cre (SEQ ID NO: 8) having the loxP target site of SEQ ID NO: 7 as shown in FIG. 1A. In cases of a non-naturally DRE, such as an evolved recombinase, the first target site is the target site on which the evolved recombinase has been evolved.

According to a preferred embodiment of the invention, the half-sites in the target site (HSA and HSB) are reverse complementary to each other, as exemplarily shown for the loxP target site in FIG. 1A.

According to a further preferred embodiment, the HSA and the HSB each have a length of between 11 and 15 nucleotides, and preferably of 13 nucleotides. According to a further preferred embodiment, the spacer in the target site has a length of between 6 and 10 nucleotides, and preferably 8 nucleotides.

The mutated sequences encoding the variant DREs, such as the PCR products of the error-prone PCR reaction, are introduced (e.g. cloned) into a suitable expression vector. This can be done by conventional methods such as digesting the coding nucleic acid using suitable restriction enzymes and ligating the coding nucleic acid into the expression vector. The expression vector to be used for the library of expression vectors can be any expression vector considered useful by the person of ordinary skill in the art. A preferred expression vector to be used in the context of the present invention is the pEVO expression vector described in Buchholz and Stewart 2001, as well as the vectors as depicted in FIGS. 1B and 1C.

The expression vector comprises at least two regions. In the first region, the expression vector comprises the nucleic acid sequence encoding the DRE variant operably linked to an expression control sequence. The expression control sequence is not particularly limited and the skilled person may readily determine suitable expression control sequence useful for the method of the present invention. The first region may further comprise a unique molecular identifier (UMI) for associating an identification means to each variant DRE. The term “unique molecular identifier” or “UMI” as used herein denotes a type of molecular barcoding. Molecular barcodes are short sequences to uniquely tag a molecule in a sample library. UMIs are known to the skilled person and are described in detail in Zurek et al., 2020, or Karst et al., 2021, both of which are herein incorporated by reference in their entirety. According to a preferred embodiment of the present invention, a UMI is an oligonucleotide comprising at least 5, at least 6, at least 7, at least 8, at least 9, at least 10, at least 20, at least 30, at least 40, at least 50, at least 60, at least 70, at least 80, at least 90 or at least 100 random nucleotides. According to a particularly preferred embodiment, a UMI is an oligonucleotide comprising at least 50 random nucleotides. A UMI may preferably form part of a UMI-tag, which may further comprise one or more sequences down-stream and/or upstream of the random nucleotides of the UMI (e.g. flanking the random nucleotides), which sequences may serve as one or more primer binding sites. A UMI-tag may further comprise one or more and preferably at least two restriction sites preferably down-stream and/or upstream of the random nucleotides of the UMI. According to a preferred embodiment, a UMI-tag comprises a first primer binding site, a first restriction site, the random nucleotides of the UMI, a second restriction site, and a second primer binding site.

In addition to the first region, the expression vector further comprises a second region comprising a nucleotide sequence comprising in 5′ to 3′ direction a first target site, an insert nucleotide sequence (INS) of a length at least 1 nucleotide and a second target site. The second region on the expression vector is separated from the first region in that both regions do not overlap. The INS has preferably a length of at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, or at least about 200 nucleotides, preferably at least about 6, 12 or 24 nucleotides, more preferably at least 6 nucleotides. The target sites in the second region each comprise in 5′ to 3′ direction a half-site A (HSA), a 1. variant spacer (vSpacer or 1. vSpacer), and a half-site B (HSB). The 1. vSpacer differs by at least one nucleotide from the Spacer of the target site of the nvDRE and any previous variant spacer, such as by at least one, two, three, four, five, six, seven, eight, nine or ten nucleotides. According to a preferred embodiment, the first target site and the second target site in the second region on the vector are identical to each other.

As for the first target site, the half-sites (HSA and HSB) of the target sites on the second region of the vector are preferably reverse complementary to each other. According to a further preferred embodiment, the HSA and the HSB each have a length of between 11 and 15 nucleotides, and preferably of 13 nucleotides. According to a further preferred embodiment, the 1. vSpacer (and any 1+n. vSpacer) in the target site has a length of between 6 and 10 nucleotides, and preferably 8 nucleotides.

Since the present invention is directed to a method for producing a spacer-specific DNA recombining enzyme, the nucleotide sequences of the half-sites HSA and HSB of the target sites in the second region of the vector are preferably identical to the half-sites of the target site to which the nvDRE binds. In other words, only the sequence of the vSpacer (and any 1+n. vSpacer) differs from the sequence of the Spacer, and the sequences of the HSA and HSB remain unchanged compared to the target site of the nvDRE.

The library of expression vectors containing the nucleotide sequences encoding the vDREs and the target sites are subsequently introduced into suitable (host) cells. The method according to the invention can be performed using eukaryotic and prokaryotic (host) cells. Preferred prokaryotic cells are bacterial cells. Particularly preferred prokaryotic cells are cells of Escherichia coli. Preferred eukaryotic cells are yeast cells (preferably Saccharomyces cerevisiae), insect cells, non-insect invertebrate cells, amphibian cells, or mammalian cells (preferably somatic or pluripotent stem cells, including embryonic stem cells and other pluripotent stem cells, like induced pluripotent stem cells, and other native cells or established cell lines, including NIH3T3, CHO, HeLa, HEK293, hiPS). According to a particularly preferred embodiment, the (host) cells are XL-1 Blue E. coli cells, and the ligated plasmids are introduced via electroporation of the cells. The skilled person is well aware about alternative suitable methods for introducing a ligated plasmid into a host cell for subsequent expression of the encoded protein. According to one embodiment, the cell is not a human germ cell. The host cells carrying the library of expression vectors are cultured to allow the expression of the encoded DRE variants. The culturing conditions are not particularly limited and will be selected by the skilled person based on the host cells used. For example, in case of using XL-1 Blue E. coli cells, it is preferred to culture the transformed bacteria in LB medium at 37° C. Conditions for introducing expression of the encoded LSR variants also depend on the host cells and plasmid vectors used. In the case of using pEVO expression vectors and XL-1 Blue E. coli cells, expression can be induced by adding e.g. arabinose to the culture medium.

The expressed vDREs—if active on the respective target sites—actively excise the portion (i.e. the nucleotide sequence) between the two target sites on the expression vector, which includes the insert nucleotide sequence (INS).

After the expression, DNA of the cultures of host cells is optionally isolated using any suitable method known to the person or ordinary skill in the art. According to a preferred embodiment, the vector DNA is isolated.

In a subsequent step, the method of the invention comprises step e) of determining whether the vDRE shows activity on the target sites of at least one expression vector. According to an alternative embodiment, step e) comprises determining whether at least a part of the INS has been excised in at least one expression vector. It will be appreciated that this step can be performed on the isolated DNA, or on the culture of cells or individual cells. It is this not mandatory to isolate DNA from the host cells. According to a preferred embodiment, step e) involves performing restriction digestion of the expression vector with one or more restriction enzymes that cleave the INS. Preferably, the restriction digestion is performed on the sequence of the expression vector comprising the two target sites, followed by analysis of the digestion fragments. The restriction enzyme(s) is preferably selected so as to excise the portion of the DNA encoding the vDRE, leading to a larger fragment including the sequence in between the two target sites in case of no excision by the vDRE (that is the vDRE is inactive on the target sites), and to a smaller fragment in case of an excision reaction between the two target sites (that is the vDRE is active on the target sites). The size difference can be visualized for example in agarose gel electrophoresis. The visualizations can then be analysed for the relative amount of large and small fragments, allowing the calculation of a percentage value for recombined and non-recombined plasmids. Band intensity values can for example be divided by the combined values of the recombined and non- recombined bands to determine the fraction of recombined DNA, which can be converted to a percentage value by multiplying with 100.

According to an alternative embodiment, step e) involves performing PCR on the host cell or the isolated DNA with a first primer specifically hybridizing 5′ of or partially overlapping or fully overlapping with the first target site, and a second primer specifically hybridizing 3′ of or partially overlapping or fully overlapping with the second target site. This embodiment may optionally further comprise sequencing of the PCR product. Based on the size and/or sequence of the PCR product, it may be determined whether, and if, to what extend an excision of the region between the two target sites on the second region of the vector including the INS took place. In cases of an excision reaction by an active DRE, the sequence will be shorter, lacking the excised sequence between the two target sites.

Also, the isolated DNA can be analysed for activity of the vDRE on the target sites. For example, at least the respective portion of the vector encoding the two target sites and the INS in between the two target sites can be sequenced. The sequencing preferably involves sequencing of at least the second region of the expression vector. The sequencing may also include the first region of the expression vector encoding the vDRE.

According to one embodiment of the invention, those variants that turned out not to be able to excise at least a part of the INS between the two target sites are preferably removed from the library. Thus, according to one embodiment, the method further comprises the step of removing inactive variants of the 1. or 1+n. vDREs from the 1. library and 1+n. libraries of expression vectors. An exemplary method of removing inactive vDREs is analogous to the restriction digestion method for determining whether the vDRE is active on the respective target sites. The restriction enzyme(s) digest the plasmid between the target sites encoded in the second region of the vector. In the absence of an excision reaction, the restriction enzyme will cut the vector in between the two target sites and the vector will be linearized. If the vDRE is active, the respective portion between the two target sites will be excised and no restriction digestion that could linearize the plasmid will occur. PCR primers can be selected so as to allow amplification of the first region comprising the sequence encoding the vDRE and the second region comprising the two target sites flanking the INS, wherein the PCR primers point at each other. If an excision reaction took place, any restriction site is removed and the vector stays intact, preserving correct orientation the PCR primers and thus amplification of a product including the first and the second regions on the plasmid. This PCR product can then be used for the next evolution cycle in that it serves as basis for introducing further mutations e.g. by error-prone PCR as described for step a) to generate second, third and further (1+n.) vDREs.

The recombination activity or efficiency of a DRE (exemplified herein as recombination rate in percent) can be determined using e.g. the plasmid-based recombination assay as described in general terms in the detailed description, and in more detail in the example section. In general, recombination activity or efficiency can be calculated from agarose gel band intensities or from deep sequencing read counts. If calculating from agarose gel band intensities, the following formula can be used: [(recombined band intensity)/(recombined band intensity +non-recombined band intensity)]×100. If calculating from deep sequencing, the following formula can be used: [(count of recombined reads)/(count of recombined reads+count of non-recombined reads)]×100. In more general terms, recombination activity or efficiency can be calculated by dividing the recombination events by the total of recombined and non-recombined events.

According to one embodiment, the recombination efficiency/activity of the vDREs differs from the recombination efficiency/activity of the nvDRE (and optionally of previous vDREs) by at least 1.5-fold. According to a preferred embodiment, the recombination efficiency/activity of a vDRE is preferably increased compared to the recombination efficiency of the nvDRE and any previous vDRE. More preferably, the recombination efficiency/activity of a vDRE is increased compared to the recombination efficiency/activity of the nvDRE and any previous vDRE by at least at least 10%, 20%, 30%, 40%, 50%, 60%, 70%, 80%, 90%, 100%, 150%, 200%, 250%, or at least 300%.

According to one embodiment, a vDRE is considered to be active on a respective target site if its activity is the same or higher as the activity of the nvDRE or the previous vDRE, or if its activity is reduced by less than 10%, 15%, 20%, 25%, 30%, 35%, 40%, 45%, or 50% compared to the nvDRE or the previous vDRE, preferably by less than 25%.

According to one embodiment, step e) optionally further comprises repeating steps a) to e) until a vDRE is produced that shows activity on the target sites, preferably an increase in recombination activity as described herein. According to an alternative embodiment, step e) optionally further comprises repeating steps a) to e) until a vDRE is produced that excises at least part of the INS.

According to one embodiment, the optional step of repeating steps a) to e) is performed for at least two times. That is, steps a) to e) are preferably repeated twice, three times, four times, five times, six times, seven times, eight times, nine times, ten times, eleven times, twelve times, 13×, 14×, 15×, 16×, 17×, 18×, 19×, 20×, 21×, 22×, 23×, 24×, 25×, 26×, 27×, 28×, 29×, 30× or more, wherein x denotes times.

According to a further embodiment, the method of the invention further comprises the steps of:
f) providing 1+n. libraries of expression vectors encoding a plurality of 1+n. variants of the vDREs (1+n. vDREs), wherein the 1+n. vDREs comprise one or more amino acid modifications in comparison to the n. vDREs, wherein each expression vector comprises:
   (i) a first region comprising a nucleotide sequence encoding one of the 1+n. vDREs from among the plurality of the 1+n. vDREs operably linked to an expression control sequence, and
   (ii) a second region comprising a nucleotide sequence comprising a first and a second target site 5′ and 3′, respectively, of an insert nucleotide sequence (INS) of a length at least 1 nucleotide, wherein each of the first and second target site comprises the HSA, a 1+n. variant spacer (1+n. vSpacer) and the HSB, wherein the 1+n. vSpacer differs by at least one nucleotide from the 1. vSpacer;
g) introducing the 1+n. libraries of expression vectors into host cells;
h) expressing the plurality of 1+n. vDREs in the host cells;
i) isolating DNA from the host cells; and
j) determining whether the 1+n. vDREs show activity on the target sites of at least one expression vector, wherein n≥1.

The features and embodiments of this further embodiment of the present method are analogous to the features and embodiments described for the method of the invention above. The term “1+n. vDRE” and likewise “1+n. vSpacer” and “1+n. libraries” each denotes a vDRE following first variant DRE (or 1. vDRE), each vSpacer following first variant Spacer (or 1. vSpacer), and each library following the first library (1. Library), respectively. In other words, the term “1+n. vDRE” denotes the variant DRE derived from the 1. variant DRE, and depending on the integer chosen for n, also each following variant DRE, such as e.g. the second variant DRE if n=1 derived from the first variant DRE, the third variant DRE if n=2 derived from the second variant DRE, the fourth variant DRE if n=3 derived from the third variant DRE and so on. The term “1+n. vSpacer” denotes the variant Spacer derived from the 1. variant Spacer, and depending on the integer chosen for n, also each following variant Spacer, such as e.g. the second variant Spacer if n=1 derived from the first variant Spacer, the third variant Spacer if n=2 derived from the second variant Spacer, the fourth variant Spacer if n=3 derived from the third variant Spacer and so on. The term “1+n. libraries” denotes the library derived from the 1. library, and depending on the integer chosen for n, also each following library, such as e.g. the second library if n=1 derived from the first library, the third library if n=2 derived from the second library, the fourth library if n=3 derived from the third library and so on.

Therefore, the term “1+n.” as used herein generally refers to a number of variants generated in addition to the first variant, denoted as 1. variant. With n being an integer, the number of variants increases in addition to the first variant depending on the choice of n. The same also applies to the libraries of expression vectors encoding a plurality of variants of a DRE. The term “1+n. libraries” thus refer to the number of libraries determined by the choice of n in addition to the first library (1. library) and includes for example 2 libraries if n=1, three libraries if n=2 and so on.

In accordance with the present invention, a variant-be it the first (1.), second, third or any subsequent variant (1+n. variant)-of the Spacer differs from a known, initial, starting or previous spacer in at least one nucleotide. That is, one or more mutations are introduced into the nucleic acid sequence of the spacer, which preferably leads to the same number of nucleotides in the spacer. Thus, according to a particularly preferred embodiment, the variant Spacer differs from the previous spacer (be it the known, initial or starting spacer such as the Spacer, or a preceding variant spacer) in at least one, two, three, four, five, six, seven, eight, nine or ten nucleotides. Most preferably, the variant spacer differs from the previous spacer in between 1 and 5 nucleotides, more preferably in between 1 and 3 nucleotides. It is to be understood that the term “Spacer” denotes the starting spacer, the term “first spacer variant” or “1. variant(s) of a spacer” or “1. vSpacer” denotes the first variant of the spacer derived from Spacer, and the term “further variant(s) of a spacer” or “1+n. vSpacer” denotes each following spacer variant. In other words, the term “1+n. vSpacer” denotes the variant spacer derived from the first spacer variant (1. vSpacer), and depending on the integer chosen for n, a respective following variant spacer, such as e.g. the second variant spacer if n=1 derived from the first variant spacer, the third variant spacer if n=2 derived from the second variant spacer, the fourth variant spacer if n=3 derived from the third variant spacer and so on. According to the present invention, n is an integer, preferably an integer selected between 1 and 50, more preferably between 1 and 20.

According to one embodiment of the present invention, the method steps f) to j) are repeated until a vDRE is produced that shows activity on the target sites of at least one expression vector. Alternatively, method steps f) to j) are repeated until a vDRE is produced that excises the INS.

According to a further embodiment, n is increased by 1, once a 1+n. vDRE is produced that shows activity on the target sites of at least one expression vector. Alternatively, n is increased by 1, once a 1+n. vDRE is produced that excises the INS. According to one embodiment of the invention, a variant DRE (vDRE) that excises the INS means a vDRE that shows at least 10% recombination efficiency/activity, preferably at least 20%, at least 30%, at least 40%, at least 50%, at least 60%, at least 70%, at least 80%, at least 90%, at least 100% or more, as preferably determined by an activity/efficiency assay as described herein. In accordance with the present invention, a DRE or vDRE is considered to show activity on a target site if its activity is determined to be at least 10%, preferably at least 20%, at least 30%, at least 40%, at least 50%, at least 60%, at least 70%, at least 80%, or at least 90%. The activity or efficiency of a DRE (e.g. the recombination rate in percent) can be determined using e.g. the assays as described herein.

According to an alternative embodiment, the method of the invention does not include variants of the spacer, but the vDREs are evolved on the sequence of the spacer of the first target site to e.g. improve the spacer specificity of the DRE. To this end, the method in step a) (ii) does not include a variant spacer, but the second region on the vector comprises a nucleotide sequence comprising in 5′ to 3′ direction a first target site, an insert nucleotide sequence (INS) of a length at least 1 nucleotide and a second target site, wherein each of the first and second target site comprises the HSA, a spacer and the HSB. In such cases, the spacer of the target sites in the second region of the vector are identical to the Spacer of the first target site of the nvDRE. If the half-sites of the target sites in the second region of the vector are identical to the half-sites of the first target site of the nvDRE, and the spacer of the target sites in the second region of the vector are identical to the Spacer of the first target site of the nvDRE, the target sites in the second region of the vector are identical to the first target site of the nvDRE. According to this alternative embodiment of the method of the invention, the vDREs are evolved until a desired activity of the vDREs is achieved on the target sites, such as an increase in activity by at least 10%, 20%, 30%, 40%, 50%, 60%, 70%, 80%, 90%, 100%, 150%, 200%, 250%, or at least 300%. In the case of an improvement in spacer specificity, the vDREs evolved by the alternative method of the invention preferably show a recombination efficiency/activity that differs from the recombination efficiency/activity of the nvDRE by at least 10%, 20%, 30%, 40%, 50%, 60%, 70%, 80%, 90%, 100%, 150%, 200%, 250%, or at least 300%. Thus, in accordance with this alternative method of the present invention, the method comprises the steps of:
a) providing a 1. library of expression vectors encoding a plurality of 1. variants of a DRE (vDRE), wherein the amino acid sequences of the 1. vDREs comprise one or more amino acid modifications in comparison to a non-variant DRE (nvDRE) from which the 1. vDREs are derived, wherein the nvDRE binds to a first target site comprising in 5′ to 3′ direction a half-site A (HSA), a spacer (Spacer) and a half-site B (HSB), wherein each expression vector comprises:
   (i) a first region comprising a nucleotide sequence encoding one of the 1. vDREs from among the plurality of 1. vDREs operably linked to an expression control sequence, and
   (ii) a second region comprising a nucleotide sequence comprising in 5′ to 3′ direction a first target site, an insert nucleotide sequence (INS) of a length at least 1 nucleotide and a second target site, wherein each of the first and second target site comprises the HSA, the spacer (Spacer) and the HSB;
b) introducing the library of expression vectors into host cells;
c) expressing the plurality of 1. vDREs in the host cells;
d) isolating DNA from the host cells; and
e) determining whether the 1. vDRE shows activity on the target sites of at least one expression vector. The method optionally further comprises repeating steps a) to e) until a 1. vDRE is produced that shows activity on the target sites of at least one expression vector.

The features and embodiments of this alternative method of the present invention are analogous to the features and embodiments described for the method of the invention above.

According to a further embodiment, the method of the present invention further comprises the step of determining the number of DNA modification events for or in each vaDRE. This step identifies which amino acids in the vDRE have been modified compared to the nvDRE or to the previous vDRE from which the vDRE is derived.

According to a further embodiment, the method of the present invention further comprises the step of determining an activity for each vDRE obtained by the method of the present invention. In particular, the activity can be determined for those vDREs that have been found to excise at least part of the INS on the expression vector between the two target sites.

The present invention is also directed to vDREs obtained by the method of the present invention. These vDREs differ in at least one amino acid from a nvDRE, such as in at least two, three, four, five, six, seven, eight, nine, ten, or more amino acids, preferably in at least 15, 20, 25, 30, 35, 40, 45, 50, 55, 60, 65, 70, 75, 80, 85, 90, 95, 100, 150, 200, 250, 300, 350, or in at least 400 amino acids from a nvDRE. The difference between the vDRE obtained by the method of the present invention and a nvDRE can also be expressed in terms of percentage identity. Accordingly, according to one embodiment, the vDRE obtainable by the method of the present invention has a sequence identity of not more than 80%, 81%, 82%, 83%, 84%, 85%, 86%, 87%, 88%, 89%, 90%, 91%, 92%, 93%, 94%, 95%, 96%, 97%, 98%, 99%, 99.2%, 99.3%, 99.4%, 99.5%, 99.6%, 99.7%, 99.8%, or 99.9% to any naturally occurring DRE, such as Cre. According to a further preferred embodiment, the vDRE obtainable by the method of the present invention comprises or consists of an amino acid sequence having at least 85% sequence identity to SEQ ID NO: 13, 14, or 15. Preferably, the vDRE obtainable by the method of the present invention differs from any known DRE such as Cre in at least one amino acid. According to a particularly preferred embodiment, the vDRE obtainable by the method of the present invention comprises or consists of an amino acid sequence having at least 85% sequence identity to SEQ ID NO: 15 and differs from VCre in at least one, two, three, four, five, six, seven or eight amino acids. According to a particular preferred embodiment, the vDRE obtainable by the method of the present invention comprises or consists of the amino acid sequence of SEQ ID NO: 15.

According to a particularly preferred embodiment, the difference in the amino acid sequence is caused by one or more amino acid substitutions, i.e. the variant differs from the nonvariant in that it comprises a respective number of amino acid substitutions and thus the same overall number of amino acids as in the wildtype or the preceding variant.

According to one embodiment of the present invention, the vDRE is present as a monomer. According to a preferred embodiment of the present invention, the vDRE comprises at least two vDRE monomers, i.e. is in a dimeric form (dimer). Such a dimer can comprise two monomers of the same type (i.e. two identical vDRE monomers, homodimer) or two monomers of a different type (i.e. two different vDRE monomers, heterodimer). According to a further preferred embodiment of the present invention, the vDRE comprises at least four protein monomers, i.e. is in a tetrameric form (tetramer). Such a tetramer can comprise four monomers of the same type (i.e. four identical vDRE monomers, homotetramer), or monomers of a different type such as two, three or four different vDRE monomers (heterotetramer). According to a particularly preferred embodiment, the vDRE is present as a dimer and comprises two identical vDRE monomers.

The present invention further pertains to a nucleic acid or group of nucleic acids encoding a vDRE according to the present invention. For activation of the expression of such nucleic acid or group of nucleic acids, the nucleic acid encoding for the vDRE preferably further comprises a regulatory nucleic acid sequence, preferably a promoter region. Hence, expression of the nucleic acid encoding for the vDRE protein can be initiated or regulated by activating the regulatory nucleic acid sequence. Accordingly, one way for inducing a DNA integration of a donor nucleic acid into a cell or genome of a subject is introducing the nucleic acid sequence or group of nucleic acid sequences of the present invention into a respective subject or cell, and activating the regulatory nucleic acid sequence (preferably the promoter region) to express the gene encoding for the vDRE protein. Preferably, the regulatory nucleic acid sequence (preferably the promoter region) is either introduced into a respective cell, preferably together with the sequence encoding for the vDRE protein, or the regulatory nucleic acid sequence is already present in said cell at the beginning. In the second case, merely the nucleic acid encoding for the vDRE protein is introduced into the cell (and placed under the control of the regulatory nucleic acid sequence).

The term “regulatory nucleic acid sequence” as used herein refers to gene regulatory regions of DNA. In addition to promoter regions, this term encompasses operator regions more distant from the gene as well as nucleic acid sequences that influence the expression of a gene, such as cis-elements, enhancers or silencers. The term “promoter region” as used herein refers to a nucleotide sequence on the DNA allowing a regulated expression of a gene. The promoter region allows regulated expression of the nucleic acid encoding for the respective protein. The promoter region is located at the 5′-end of the gene and thus before the RNA coding region. Both, bacterial and eukaryotic promoters are applicable for the invention.

The present invention also includes one or a plurality of nucleic acids or nucleic acid sequences or polynucleotides in which the coding sequence for the vDRE is fused in the same reading frame to a polynucleotide sequence which aids in expression and secretion of a protein from a host cell. For example, a leader sequence, which functions as a secretory sequence for controlling transport of a polypeptide from the cell, may be fused to the sequence encoding the vDRE protein. The polypeptide or protein having such a leader sequence is termed a pre-protein or a pre-proprotein and may have the leader sequence cleaved by the host cell to form the mature form of the protein. These polynucleotides may have a 5′ extended region so that it encodes a proprotein, which is the mature protein plus additional amino acid residues at the N-terminus. The expression product having such a pro-sequence is termed a pro-protein, which is an inactive form of the mature protein; however, once the pro-sequence is cleaved, an active mature protein remains. The additional sequence may also be attached to the protein and be part of the mature protein. Thus, for example, the polynucleotides of the present invention may encode polypeptides, or proteins having a pro-sequence, or proteins having both, a pro-sequence and a pre-sequence (such as a leader sequence).

The nucleic acids of the present invention may also have the coding sequence fused in frame to a marker sequence, which allows for purification of the vDRE protein of the present invention. The marker sequence may be an affinity tag or an epitope tag such as a polyhistidine tag, a streptavidin tag, an Xpress tag, a FLAG tag, a cellulose or chitin binding tag, a glutathione-S transferase tag (GST), a hemagglutinin (HA) tag, a c-myc tag or a V5 tag.

If the nucleic acid of the invention is an mRNA, in particular for use as a medicament, the delivery of mRNA therapeutics can be facilitated by the significant progress that has been achieved in maximizing the translation and stability of mRNA, preventing its immune-stimulatory activity and the development of in vivo delivery technologies. The 5′ cap and 3′ poly(A) tail are the main contributors to efficient translation and prolonged half-life of mature eukaryotic mRNAs. Incorporation of cap analogs such as ARCA (anti-reverse cap analogs) and poly (A) tail of 120-150 bp into in vitro transcribed (IVT) mRNAs has markedly improved expression of the encoded proteins and mRNA stability. New types of cap analogs, such as 1,2-dithiodiphosphate-modified caps, with resistance against RNA decapping complex, can further improve the efficiency of RNA translation. Replacing rare codons within mRNA protein-coding sequences with synonymous frequently occurring codons, so-called codon optimization, also facilitates better efficacy of protein synthesis and limits mRNA destabilization by rare codons, thus preventing accelerated degradation of the transcript. Similarly, engineering 3′ and 5′ untranslated regions (UTRs), which contain sequences responsible for recruiting RNA-binding proteins (RBPs) and miRNAs, can enhance the level of protein product. Interestingly, UTRs can be deliberately modified to encode regulatory elements (e.g., K-turn motifs and miRNA binding sites), providing a means to control RNA expression in a cell-specific manner. Some RNA base modifications such as N1-methyl-pseudouridine have not only been instrumental in masking mRNA immune-stimulatory activity but have also been shown to increase mRNA translation by enhancing translation initiation. In addition to their observed effects on protein translation, base modifications and codon optimization affect the secondary structure of mRNA, which in turn influences its translation. Respective modifications of the nucleic acid molecules of the invention are also contemplated by the invention.

The RNA or plurality of RNAs preferably encode the vDRE enzyme of the present invention or any of its subunits. Specific methods for delivering and expressing nucleic acids and specifically RNAs are disclosed e.g. in EP2590676 and EP3115064, which are herein incorporated by reference. The RNA may be present in a particle and is preferably self-replicating. After in vivo administration of the particles, RNA is released from the particles and is translated inside a cell to provide the vDRE or any of its monomeric subunits.

A self-replicating RNA molecule (replicon) can, when delivered to a vertebrate cell even without any proteins, lead to the production of multiple daughter RNAs by transcription from itself (via an antisense copy which it generates from itself). These daughter RNAs, as well as collinear sub-genomic transcripts, may be translated by themselves to provide in situ expression of an encoded polypeptide, or may be transcribed to provide further transcripts with the same sense as the delivered RNA which are translated to provide in situ expression of the polypeptide. The overall results of this sequence of transcriptions is a huge amplification in the number of the introduced replicon RNAs and so the encoded polypeptide becomes a major polypeptide product of the cells.

A preferred self-replicating RNA molecule encodes (i) an RNA-dependent RNA polymerase which can transcribe RNA from the self-replicating RNA molecule, and (ii) a vDRE protein of the present invention. The polymerase can be an alphavirus replicase e.g. comprising one or more of alphavirus proteins nsP1, nsP2, nsP3 and nsP4. It is preferred that the self-replicating RNA molecules of the invention do not encode alphavirus structural proteins. Thus, a preferred self-replicating RNA can lead to the production of genomic RNA copies of itself in a cell, but not to the production of RNA-containing virions. A self-replicating RNA molecule useful in the context of the present invention may have two open reading frames. The first (5′) open reading frame encodes a replicase, and the second (3′) open reading frame encodes a polypeptide of the present invention. In some embodiments, the RNA may have additional (e.g. downstream) open reading frames e.g. for further encoding accessory polypeptides.

Such RNA is particularly suitable for the general use in gene therapy, and specifically for use in the treatment of genetic disorders or diseases.

The present invention further provides an expression vector comprising the nucleic acid according to the present invention. The expression vector is preferably a pEVO vector as described in Buchholz and Stewart, 2001, more preferably the variant pEVO vector described herein and as shown ion FIGS. 1B and 1C. According to a particularly preferred embodiment, the vector comprises a nucleic acid sequence encoding a protein having at least 85% identity to any one of SEQ ID NOs: 13, 14 or 15.

The present invention further provides a cell or host cell comprising the vector or the nucleic acid or group of nucleic acids according to the present invention. The skilled person readily identifies suitable host cells, which may be eukaryotic or prokaryotic. Preferred prokaryotic cells are bacterial cells. Particularly preferred prokaryotic cells are cells of Escherichia coli. Preferred eukaryotic cells are yeast cells (preferably Saccharomyces cerevisiae), insect cells, non-insect invertebrate cells, amphibian cells, or mammalian cells (preferably somatic or pluripotent stem cells, including embryonic stem cells and other pluripotent stem cells, like induced pluripotent stem cells, and other native cells or established cell lines, including NIH3T3, CHO, HeLa, HEK293, hiPS). According to a particularly preferred embodiment, the host cells are XL-1 Blue E. coli cells.

The present invention further provides a system for integrating a donor nucleic acid (e.g. DNA) into a target nucleic acid such as into the genome of a subject or cell. The system comprises a polypeptide comprising a vDRE according to the present invention, or a nucleic acid or group of nucleic acids encoding the same, and a donor nucleic acid to be inserted into the target nucleic acid. The present invention also provides a system for exchanging a nucleic acid sequence of interest in the genome of a subject or cell for a different nucleic acid sequence, wherein the cell does not include cells of the human germ line. The system comprises a polypeptide comprising a vDRE according to the present invention, or a nucleic acid or group of nucleic acids encoding the same, and a nucleic acid sequence differing from the nucleic acid sequence to be exchanged, which is preferably inserted at the position of the nucleic acid sequence to be exchanged in the genome of a subject or cell.

The system according to the invention is applicable for use in combination with other DRE systems and thus may become a particular valuable tool for genetic experiments where multiple DREs are required simultaneously or sequentially.

The present invention also provides a pharmaceutical composition comprising the vDRE according to the present invention, the one or more nucleic acids according to the present invention, or the vector according to the present invention, and optionally a pharmaceutically acceptable carrier. The pharmaceutical composition may be in any form that is suitable for the selected mode of administration.

In one embodiment, the pharmaceutical composition of the present invention is administered parenterally.

The phrases “parenteral administration” and “administered parenterally” as used herein means modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection and infusion.

The therapeutically active agents in the pharmaceutical composition of the invention include but are not limited to the vDRE according to the present invention, the one or more nucleic acids according to the present invention, or the vector according to the present invention. According to a preferred embodiment, the active agent in the pharmaceutical composition is the vDRE according to the present invention and in particular a vDRE having at least 85% identity to any one of SEQ ID NOs: 13, 14 or 15. The pharmaceutical composition comprising the therapeutically active agent can be administered as sole pharmaceutical composition, or in combination with other active agents, in a unit administration form, as a mixture with conventional pharmaceutical supports, to animals and human beings.

In further embodiments, the pharmaceutical composition contains one or more carriers (also termed vehicles) which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid. It must be stable under the conditions of manufacture and storage and must be preserved against the contaminating action of microorganisms, such as bacteria and fungi.

According to a further aspect, the present invention provides the use of a vDRE according to the present invention and in particular the use of a protein having at least 85% identity to any one of SEQ ID NOs: 13, 14 or 15 for exchanging a nucleic acid sequence of interest in the genome of a subject or cell for a different nucleic acid sequence, wherein the cell does not include cells of the human germ line. According to a further aspect, the present invention provides the use of a vDRE according to the present invention for integrating a donor nucleic acid sequence of interest into the genome of a subject or cell, wherein the cell does not include cells of the human germ line. According to a particularly preferred embodiment, the donor nucleic acid encodes a polypeptide or protein of interest and is integrated into the target nucleic acid such that the polypeptide or protein of interest is expressed when the nucleic acid is transcribed and translated. The donor nucleic acid preferably comprises at least one target site of the vDRE.

According to a further aspect, the present invention provides the use of the nucleic acid or group of nucleic acids of the present invention for exchanging a nucleic acid sequence of interest in the genome of a subject or cell for a different nucleic acid sequence, or for integrating a donor nucleic acid into a target nucleic acid such as into the genome of a subject or cell. According to a particularly preferred embodiment, the donor nucleic acid encodes a polypeptide or protein of interest and is integrated into the target nucleic acid such that the polypeptide or protein of interest is expressed when the nucleic acid is transcribed and translated. The donor nucleic acid preferably comprises at least one target site of the vDRE.

According to a further aspect, the present invention provides the use of the expression vector or the pharmaceutical composition of the present invention for exchanging a nucleic acid sequence of interest in the genome of a subject or cell for a different nucleic acid sequence, or for integrating a donor nucleic acid into a target nucleic acid such as into the genome of a subject or cell. According to a particularly preferred embodiment, the donor nucleic acid encodes a polypeptide or protein of interest and is integrated into the target nucleic acid such that the polypeptide or protein of interest is expressed when the nucleic acid is transcribed and translated. The donor nucleic acid preferably comprises at least one target site of the vDRE. According to a further aspect, the present invention provides the use of the system of the present invention for exchanging a nucleic acid sequence of interest in the genome of a subject or cell for a different nucleic acid sequence, or for integrating a donor nucleic acid into a target nucleic acid such as into the genome of a subject or cell. According to a particularly preferred embodiment, the donor nucleic acid encodes a polypeptide or protein of interest and is integrated into the target nucleic acid such that the polypeptide or protein of interest is expressed when the nucleic acid is transcribed and translated. The donor nucleic acid preferably comprises at least one target site of the vDRE.

The present invention further provides vDRE according to the present invention, the nucleic acid or group of nucleic acids of the present invention, the expression vector of the present invention, the pharmaceutical composition of the present invention, or the system of the present invention for use in medicine.

The present invention further provides the vDRE according to the present invention, the nucleic acid or group of nucleic acids of the present invention, the expression vector of the present invention, the pharmaceutical composition of the present invention, or the system of the present invention for use in the treatment of a genetic disease or disorder. The genetic disease or disorder is not particularly limited and includes e.g. any genetic disease or disorder that can be treated by integrating a nucleic acid sequence or plurality of nucleic acid sequences into the genome of a respective patient that encodes one or more proteins or polypeptides that aid in treating the genetic disease or disorder. The genetic disease or disorder may be further treated by exchanging a nucleic acid sequence or plurality of nucleic acid sequences in the genome of a respective patient by a different nucleic acid sequence or plurality of nucleic acid sequences. A preferred genetic disease or disorder is a monogenetic disease or disorder.

According to one embodiment, a (host) cell within the meaning of the invention is preferably a naturally occurring cell or a cell line (optionally transformed or genetically modified) that comprises at least one vector according to the invention or a nucleic acid according to the invention recombinantly, as described above. Thereby, the invention includes transient transfectants (e.g. by mRNA injection) or host cells that include at least one expression vector according to the invention as a plasmid or artificial chromosome, as well as host cells in which an expression vector according to the invention is stably integrated into the genome of said host cell.

Further provided are kits comprising a therapeutically active agent as described herein. In one embodiment, the kit provides the therapeutically active agents prepared in one or more unitary dosage forms ready for administration to a subject, for example in a preloaded syringe or in an ampoule. In another embodiment, the therapeutically active agents are provided in a lyophilized form.

Using the present invention, it is possible to (i) integrate a donor nucleic acid, preferably DNA, into a target nucleic acid, preferably DNA, which target nucleic acid can be present in a host organism, such as mammals, or to (ii) exchange a nucleic acid of a patient or a cell by a different nucleic acid. Therefore, the present invention also includes a host organism comprising the integrated or exchanged nucleic acid. According to one embodiment, the host organism is not a human. Also provided are host organisms which comprise a vector according to the invention or a nucleic acid according to the invention as described above that is, respectively, stably integrated into the genome of the host organism or individual cells of the host organism. Preferred host organisms according to the present invention are plants, invertebrates and vertebrates, particularly Bovidae, Drosophila melanogaster, Caenorhabditis elegans, Xenopus laevis, medaka, zebrafish, or Mus musculus, or embryos of these organisms.

The present invention further pertains to a method for integrating a donor nucleic acid into a target nucleic acid such as into the genome of a subject or cell. The method comprises the steps of (i) providing the vDRE according to the present invention, the nucleic acid or group of nucleic acids of the present invention, the expression vector of the present invention, the pharmaceutical composition of the present invention, or the system of the present invention, and a donor nucleic acid, optionally allowing the vDRE to be expressed, and (ii) allowing the vDRE to integrate the donor nucleic acid into the target nucleic acid.

The present invention also pertains to a method for exchanging a nucleic acid such as a nucleic acid in the genome of a subject or cell by a different nucleic acid. The method comprises the steps of (i) providing the vDRE according to the present invention, the nucleic acid or group of nucleic acids of the present invention, the expression vector of the present invention, the pharmaceutical composition of the present invention, or the system of the present invention, and the different nucleic acid that shall replace the nucleic acid in the genome of the subject or the cell, optionally allowing the vDRE to be expressed, and (ii) allowing the vDRE to exchange the nucleic acid in the genome of the subject or cell and insert the different nucleic acid.

The present invention further pertains to a method for treating a genetic disease or disorder in a subject by integrating a donor nucleic acid into a target nucleic acid of the subject, or by exchanging a nucleic acid present in the genome of a subject or a cell. In the case of an integration, the method comprises the steps of (i) providing the vDRE according to the present invention, the nucleic acid or group of nucleic acids of the present invention, the expression vector of the present invention, the pharmaceutical composition of the present invention, or the system of the present invention, in a therapeutically effective amount, and a donor nucleic acid, optionally allowing the vDRE to be expressed, and (ii) allowing the vDRE to integrate the donor nucleic acid into the target nucleic acid. In the case of an exchange, the method comprises the steps of (i) providing the vDRE according to the present invention, the nucleic acid or group of nucleic acids of the present invention, the expression vector of the present invention, the pharmaceutical composition of the present invention, or the system of the present invention, and a different nucleic acid that shall replace the nucleic acid in the genome of the subject or the cell, optionally allowing the vDRE to be expressed, and (ii) allowing the vDRE to exchange the nucleic acid in the genome of the subject or cell and insert the different nucleic acid.

The present invention further pertains to the following items:

Item 1: A Method for generating a spacer-specific DNA recombining enzyme (DRE), the method comprising the steps of:
a) providing a 1. library of expression vectors encoding a plurality of 1. variants of a DRE (vDRE), wherein the amino acid sequences of the 1. vDREs comprise one or more amino acid modifications in comparison to a non-variant DRE (nvDRE) from which the 1. vDREs are derived, wherein the nvDRE binds to a first target site comprising in 5′ to 3′ direction a half-site A (HSA), a spacer (Spacer) and a half-site B (HSB), wherein each expression vector comprises:
   (i) a first region comprising a nucleotide sequence encoding one of the 1. vDREs from among the plurality of 1. vDREs operably linked to an expression control sequence, and
   (ii) a second region comprising a nucleotide sequence comprising in 5′ to 3′ direction a first target site, an insert nucleotide sequence (INS) of a length at least 1 nucleotide and a second target site, wherein each of the first and second target site comprises the HSA, a 1. variant spacer (vSpacer) and the HSB, wherein the vSpacer differs by at least one nucleotide from the Spacer;
b) introducing the library of expression vectors into host cells;
c) expressing the plurality of 1. vDREs in the host cells;
d) optionally isolating DNA from the host cells; and
e) determining whether the 1. vDRE shows activity on the target sites of at least one expression vector;
   optionally repeating steps a) to e) until a 1. vDRE is produced that shows activity on the target sites of at least one expression vector.

Item 2. The method according to item 1, further comprising the steps of:
f) providing 1+n. libraries of expression vectors encoding a plurality of 1+n. variants of the vDREs (1+n. vDREs), wherein the 1+n. vDREs comprise one or more amino acid modifications in comparison to the n. vDREs, wherein each expression vector comprises:
   (i) a first region comprising a nucleotide sequence encoding one of the 1+n. vDREs from among the plurality of the 1+n. vDREs operably linked to an expression control sequence, and
   (ii) a second region comprising a nucleotide sequence comprising a first and a second target site 5′ and 3′, respectively, of an insert nucleotide sequence (INS) of a length at least 1 nucleotide, wherein each of the first and second target site comprises the HSA, a 1+n. variant spacer (1+n. vSpacer) and the HSB, wherein the 1+n. vSpacer differs by at least one nucleotide from the 1. vSpacer;
g) introducing the 1+n. libraries of expression vectors into host cells;
h) expressing the plurality of 1+n. vDREs in the host cells;
i) isolating DNA from the host cells; and
j) determining whether the 1+n. vDREs show activity on the target sites of at least one expression vector, wherein n ≥ 1.

Item 3: The method of item 2, further comprising repeating steps f) to j) until a 1+n. vDRE is produced that excises the INS.

Item 4: The method of item 2, wherein n is increased by 1 once a 1+n. vDRE is produced that excises the INS.

Item 5: The method according to any one of items 1 to 4,
wherein the Spacer and the n or 1+n. variant Spacer each have a length of between 6 and 10 nucleotides, preferably 8 nucleotides; and/or
wherein the HSA and the HSB each have a length of between 11 and 15 nucleotides, preferably 13 nucleotides.

Item 6: The method according to any one of items 1 to 5, wherein the nvDRE is a naturally occurring DRE or a variant thereof that binds to a first and second half site different from the first and second half site bound by the naturally occurring DRE, preferably a tyrosine recombinase or a large serine recombinase,
preferably a tyrosine recombinase selected from the group consisting of Cre-, Dre-, VCre-, SCre-, Vika-, lambda-Int-, F1p-, R-, Kw-, Kd-, B2-, B3-, Nigri- or Panto-recombinases, or preferably a large serine recombinase selected from the group consisting of A118, TP901, φRV1, Bxb1, φC31, R4, Wβ, Tnpx, Cp36, Dn29, Kp03, Nm60, Pa01, Si74.

Item 7: The method according to any one of items 1 to 6, wherein step e) or j) of determining whether the 1. vDRE shows activity on the target sites of at least one expression vector comprises:
i) performing PCR on the host cell of c) or h) or the isolated DNA of step d) or i) with a first primer specifically hybridizing 5′ of or partially overlapping or fully overlapping with the first target site, and a second primer specifically hybridizing 3′ of or partially overlapping or fully overlapping with the second target site, and optionally sequencing of the PCR product; or
ii) restriction digestion of the expression vector with one or more restriction enzymes that cleave the INS.

Item 8: The method according to any one of items 1 to 7, further comprising the step of removing inactive variants of the 1. or 1+n. vDREs from the library of expression vectors.

Item 9: A vDRE obtainable by the method according to any of items 1 to 8, wherein the amino acid sequence of the vDRE differs in at least one amino acid from the nvDRE, preferably wherein the vDRE comprises or consists of an amino acid sequence having at least 85% identity to any one of SEQ ID NOs: 13, 14, or 15.

Item 10: A nucleic acid or group of nucleic acids encoding a vDRE according to item 9.

Item 11: An expression vector comprising a nucleic acid or group of nucleic acids according to item 10.

Item 12: A system for (i) integrating a donor nucleic acid into a target nucleic acid, or (ii) exchanging a a nucleic acid sequence of interest in the genome of a subject or cell for a different nucleic acid sequence, the system comprising
a polypeptide comprising a vDRE according to item 9, or a nucleic acid or group of nucleic acids according to item 10; and
either (i) a donor nucleic acid to be inserted into the target nucleic acid, or (ii) a nucleic acid sequence differing from the nucleic acid sequence to be exchanged.

Item 13: A pharmaceutical composition comprising the vDRE of item 9, the nucleic acid or group of nucleic acids according to item 10, or the expression vector according to item 11, and optionally a pharmaceutically acceptable carrier.

Item 14: Use of the vDRE of item 9, the nucleic acid or group of nucleic acids of item 10, the expression vector of item 11, the system of item 12, or the pharmaceutical composition of item 13, for (i) integrating a nucleic acid sequence of interest into the genome of a subject or cell, or (ii) for exchanging a nucleic acid sequence of interest in the genome of a subject or cell for a different nucleic acid sequence, wherein the cell does not include cells of the human germ line.

Item 15: The vDRE according to item 9, the nucleic acid or group of nucleic acids of item 10, the expression vector of item 11, or the pharmaceutical composition of item 13 for use in medicine, preferably for use in the treatment of a genetic disease or disorder, more preferably wherein the genetic disease or disorder is a monogenetic disease or disorder.

Item 16: A method for treating a genetic disease or disorder in a subject by integrating a donor nucleic acid into a target nucleic acid of the subject, wherein the method comprises the steps of: (i) providing the vDRE according to the present invention, the nucleic acid or group of nucleic acids of the present invention, the expression vector of the present invention, the pharmaceutical composition of the present invention, or the system of the present invention, in a therapeutically effective amount, and a donor nucleic acid, optionally allowing the vDRE to be expressed, and (ii) allowing the vDRE to integrate the donor nucleic acid into the target nucleic acid.

Item 17: A method for treating a genetic disease or disorder in a subject by exchanging a nucleic acid present in the genome of a subject or a cell, wherein the method comprises the steps of: (i) providing the vDRE according to the present invention, the nucleic acid or group of nucleic acids of the present invention, the expression vector of the present invention, the pharmaceutical composition of the present invention, or the system of the present invention, and a different nucleic acid that shall replace the nucleic acid in the genome of the subject or the cell, optionally allowing the vDRE to be expressed, and (ii) allowing the vDRE to exchange the nucleic acid in the genome of the subject or cell and insert the different nucleic acid.

### EXAMPLES

### Materials and Methods

### Plasmid Construction

The target site library plasmid backbone (pGG) was engineered from the previously described pEVO plasmid described in Buchholz and Hauber, 2011, which was modified to replace the pEVO BglII sites with two BbsI restriction sites facing outward (FIG. 1A). BbsI restriction site (downstream “left” site) designed with the following sequence: 5′-ACACCGGGTCTTC-3′ (SEQ ID NO: 1) leaving a GTGG overhang. The BbsI restriction site (upstream “right” site) designed with the following sequence: 5′-GAAGACCTGTTTA-3′ (SEQ ID NO: 2) leaving a GTTT overhang. The recombinase to be assayed was cloned into the pGG vector using BsrGI-HF and SbfI (NEB) restriction enzymes. Expression of the recombinase is controlled by an L-arabinose inducible promoter system (araBAD). The target site library was assembled to pGG-recombinase destination vector (FIG. 1A) following manufacture recommended protocol for setup of type IIs restriction digestion and ligation reaction (Engler et al., 2008) using T4 DNA Ligase and Type IIS restriction enzyme BbsI (Thermo Scientific).

The evolution plasmids pEVO-loxSE1, pEVO-loxSE2 and pEVO-loxSE3 were generated from the previously described pEVO-loxP plasmid (Sarkar et al., 2007; Buchholz and Hauber, 2011) (FIG. 1B) modified to replace the loxP target site with the evolution target sites inserted at the restriction sites PciI and BglII (NEB; FIG. 1C).

### Plasmid-Based Recombinase Activity Assay

Successful recombination events result in excision of the DNA flanked by the lox sites. Therefore, to assay the recombination efficiency of either individual recombinase variants or recombinase libraries, a simple restriction digest is used to compare plasmid sizes in the sample (Lansing et al., 2020; Buchholz et al., 2001; Lansing et al., 2022; Karpinski et al., 2016; Sarkar et al., 2007) (FIG. 1D). The recombinase or recombinase library is assembled to either the pEVO or pGG vector containing the target site or library of target sites to be tested. The recombinase or recombinase library is cloned into the vector between the restriction sites BsrGI (NEB) and XbaI (NEB), downstream of the arabinose-inducible araBAD promoter (pBAD) (FIG. 1B, 1C). The assembled plasmids are transformed via electroporation into electrocompetent E. coli X-L1 Blue strain, recovered in SOC medium at 37° C. shaking (700 rpm) for 1 hour. Cells are plated onto LB-Cm (30 μg/mL chloramphenicol) agar to verify that the ligation was successful and also inoculated in 6 mL LB-Cm (15 μg/mL chloramphenicol) containing the desired dose of L-Arabinose to induce the expression of the recombinase overnight. The L-Arabinose concentrations of 1, 10 and 100 μg/mL were used for the recombinase activity screens. Plasmids were purified from the overnight cultures following the protocol of the Qiagen spin isolation kit (Qiagen Inc.). To visually compare the recombined and non-recombined plasmids in the sample, the purified plasmid DNA is digested using BsrGI-HF and XbaI (NEB; for assaying pEVO) or using BsrGI-HF and PacI (NEB; for assaying pGG) (FIG. 1D). The digest linearizes the DNA to easily compare the smaller recombined plasmids to the non-recombined plasmids in the sample with gel electrophoresis. Recombination efficiencies were calculated by the ratio of band intensities for each well using GelAnalyzer v23.1 for image processing (GelAnalyzer 23.1, available at www.gelanalyzer.com, by Istvan Lazar Jr., PhD and Istvan Lazar Sr., PhD, CSc). Recombination was calculated by dividing the recombined band intensity by the recombined and the non-recombined band intensity. The calculated recombination was plotted in R 4.0.3 (R Core Team, 2018) with dplyr v1.0.7 (https://dplyr.tidyverse.org/) and visualized with ggplot2 v3.3.5 (https://ggplot2.tidyverse.org/). Bacterial test digests were done in triplicates (n=3).

### Library Construction

The library was generated using silicon-based DNA synthesis from Twist Bioscience, providing full control over the sequence design for each oligo in the library. Each oligo was predefined, allowing the unique target site to be encoded twice on the same oligo. To reduce costs, synthesis time, and potential PCR bias during downstream library preparation, the oligo length was kept short. Consequently, each oligo encoded a short distance (24 nt) between the two target sequences.

Previous studies have shown that the efficiency of Cre-mediated intramolecular recombination between two directly repeated loxP sites depends on the distance between the sites, with a minimum distance of 82 bp required for efficient recombination (Hoess et al., 1985). This was considered when optimizing the screen, comparing recombination of Cre/loxP for different distances between the lox sites: 24 bp and 700 bp. It was also assessed how the recombination rate at these distances changed with increasing concentrations of Cre by using L-arabinose concentrations of 0, 1, 10, and 100 μg/mL in the E. coli plasmid-based excision assay. In agreement with previous findings, the results show that a length of 700 bp between the two lox sites was more efficient than the shorter length of 24 bp. However, the trends of increasing recombination with increasing concentrations of Cre are similar for both lengths. Additionally, sequencing of plasmid DNA from both recombined and non-recombined colonies confirmed that recombination occurred as predicted, with only one loxP site and the full stuffer sequence precisely excised from the DNA of recombined colonies. Excision of DNA between lox sites located a short distance from one another likely occurs in two steps: first, intermolecular integration followed by intramolecular excision.

The target site library for the activity screen comprised 6,000 oligonucleotides ordered as oligo pools from Twist Bioscience. Each oligo sequence is a predefined length of 120 nt encoding two copies of the unique 34 nt lox-like target sequence. Between the two targets was a 24 nt region containing two restriction sites (NdeI and PacI; NEB) used for quality control. Flanking the target sites were 14 nt primer binding sites (Primers P1, P2) to amplify the synthesized ssDNA pool for dsDNA. The design of the libraries excluded all restriction enzymes used during the assay and downstream analysis.

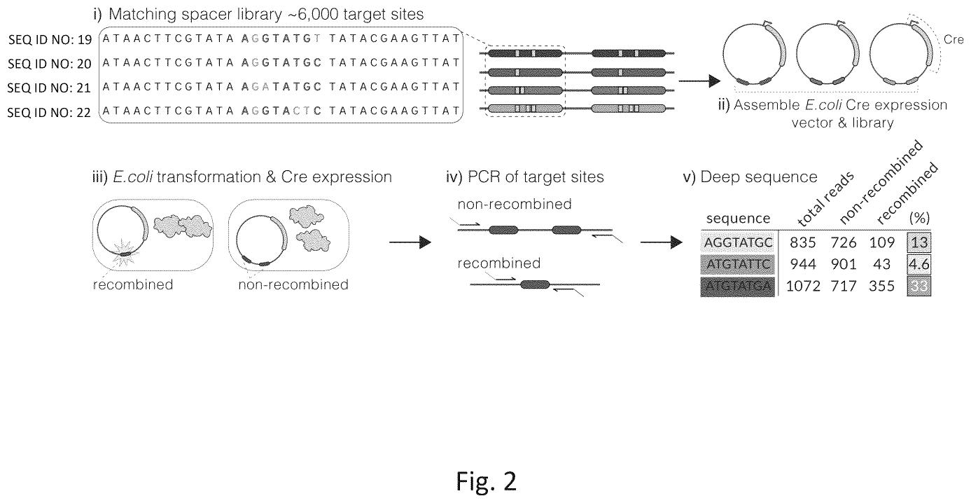

In order to understand how changes in the spacer region impact Cre recombination, the library was built on the core 8 bp spacer region while the flanking loxP half-sites were kept consistent (FIG. 2). Because the spacer characteristics contributing to specificity should be examined, the libraries were designed to contain target sites with homologous spacers; i.e., each target site variant contains the unique mutation in both spacers of the complex. Another design feature to consider was the size of the library. A library containing all spacer base combinations would consist of 65,536 different targets. Instead of having all spacer possibilities, a more rational approach was taken to minimize the size of the library, allowing for more reads per target to achieve a higher confidence interval per screen. Because the first and last bases have been reported to influence strand exchange (Lee et al., 2003; Abi-Ghanem et al., 2015; Gelato et al., 2005), the spacers were mainly designed with the 6 core nucleotides altered, which were annotate as ANNNNNNC wherein N refers to the altered position. Additionally, eight different combinations of the four core sequences with the last two and first two spacer nucleotides altered were included in the library (NNGTATNN, NNATACNN, NNCTAANN, NNCTAGNN, NNAATTNN, NNTTAANN, NNGGCCNN, NNCCGGNN). From these combinations, all fully symmetric spacers (e.g., AAAATTTT; SEQ ID NO: 3) were excluded from the library.

### Target-Site Library Preparation for Deep-Sequencing

Experiments were conducted in E. coli with Cre under the expression of an inducible promoter for a tighter control of the recombinase/target site exposure (FIG. 1A). This provided the option to adjust the expression of Cre to better distinguish functional diversity due to variation in spacer sequences. The assembled pGG-Recombinase-Library was transformed to E. coli XL1-Blue (Agilent) and the expression of the recombinase was induced with different concentrations of L-arabinose (0, 1, 10 and 100 μg/ml L-arabinose; Sigma Aldrich). After growing the cells for 14 to 16 hours in 50 ml LB and in the presence of chloramphenicol (30 mg/ml), plasmid DNA was extracted using the GeneJET Plasmid Miniprep Kit (ThermoFisher). The extracted plasmid DNA was used as a template to amplify over the target sites using primers P3 and P4, adding the indexes i5 and i7 for Illumina paired-end sequencing. PCR was performed using a high-fidelity polymerase (Herculase II Fusion DNA Polymerase, Agilent) carried out with ten PCR cycles. The kit manufacturer protocol was followed using an initial denaturing temperature of 95° C. for 3 minutes and 40 seconds per cycle, an annealing temperature of 55° C., and an extension temperature of 72°°C. for 1 minute per cycle. The resulting amplicon length is 220 bp for the non-recombined targets and 162 bp for the recombined targets. The amplicons were purified on a column using the Isolate II PCR and Gel Kit (Bioline) and quantified using the Qubit dsDNA HS Kit (ThermoFisher) according to the manufacturer's instructions. The sequencing of samples was performed using paired-end reads of 150 bases on the Illumina NovaSeq 6000.

### Validation and Quality Control of the Screen

Before analysis of the results, the quality of the screen was confirmed. Both, the Cre and RecS3 activity screen recombination outcomes were quantified with high throughput sequencing of the targets at an average sequencing depth of >1000× per target. The screen was done in triplicates to determine the consistency of the results. Pearson's correlation coefficient (R) was measured for each replicate and all target sites in the library were plotted to show relationship between the replicate and the pooled replicates recombination rates. For both the Cre and RecS3 activity screens, the value of R ranges between 0.87-0.99 indicating a positive linear relationship, and consistent recombination levels among the triplicates.

Additionally, the activities calculated from the target site screen were validated for a selection of spacers ranging from low to high Cre activity. Activities from the screen were compared to activities quantified in triplicates from plasmid-based recombination activity assay. The mean screen activity and mean plasmid-based recombination activity were calculated for the triplicates and plotted to show the relationship between activities. A Pearson's correlation coefficient was computed (R=0.97) carried out in R (version 4.3.2) showing high correlation between the two assays.

### Data Analysis

Illumina sequencing data is processed using Cutadapt (Kechin et al., 2017) and R version 4.3.2 (R Core Team, 2023) to convert to count matrices. Targets with fewer than 100 reads were discarded. Because of symmetry of the recombination reaction, alignment of the spacer either from the top or bottom strand of DNA is arbitrary. Therefore, the strand with the highest identity to the wild-type loxP or on-target sequence was considered. To facilitate a comparative analysis between distinct screens, the activity ratio for each dataset was computed by dividing the recombination rate of individual target sites by the highest recombination rate within each dataset.

Sequence logos were generated for subgroups of target sequences to compare conserved bases associated with the subgroup. Although the generated sequence logos were normalized to the number of sequences, the subgroups that were compared always consisted of the same number of sequences (e.g. subgroup A has n=10 sequences and subgroup B has n=10 sequences) in order to avoid effects from sample size.

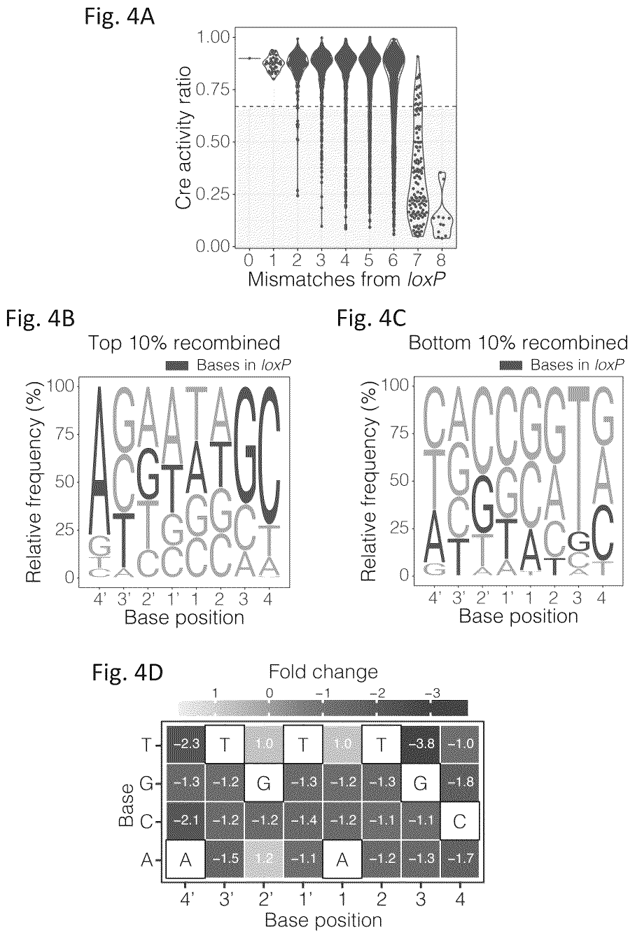

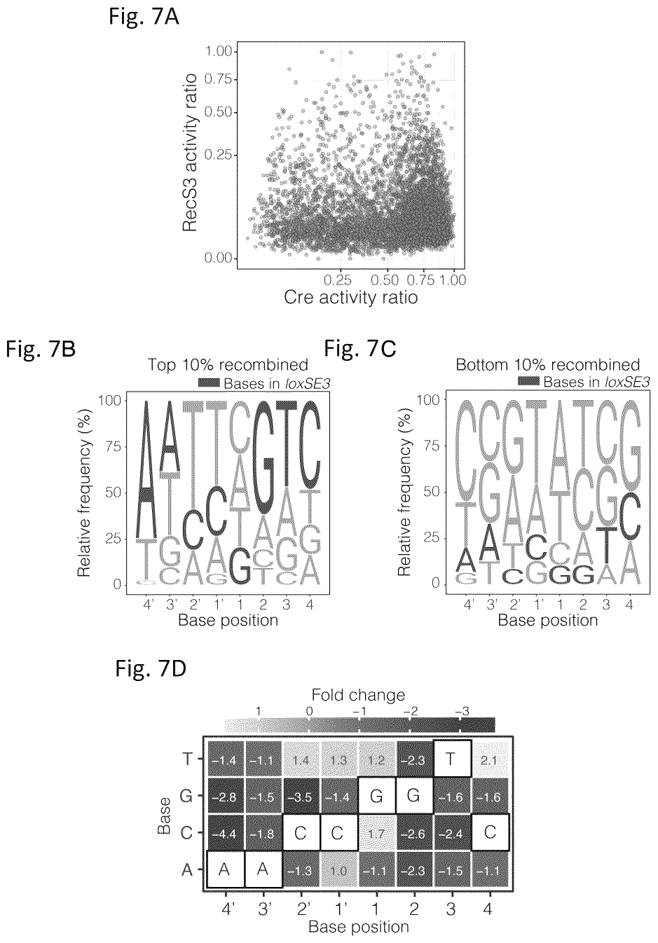

At each position in the alignment of spacer sequences, the logo plot represents the relative frequency of each base, with the height of each base proportional to its relative frequency (FIG. 4B, 4C and FIG. 7B, 7C). The plots highlight bases that have an observed frequency higher than their expected frequency (frequency in library). Standard logo base height represents observed frequencies p=(pA, pC, pG, pT) compared with a uniform background, q=(0.25, 0.25, 0.25, 0.25). Logos calculated from library subgroups (i.e., top 10% recombined or bottom 10% recombined) were normalized to the library frequency. This normalization is referred to as relative frequency. The relative frequencies were calculated by first calculating the base frequencies for each position in the subgroup (freq. subgroup) and in the library (freq. library).

$$freq . subgroup = \frac{number of occurences of base at position}{number of sequences in subgroup}$$

$$freq . library = \frac{number of occurences of base at position}{number of sequences in library}$$

The calculated frequencies were then used for relative frequency.

$$Normalized relative frequency = ( freq . subgroup ) \times ( 1 - ( freq . library )$$

The logos were plotted with R package ggseqlogo (Wagih 2017).

Effects of individual base changes on recombination levels of different target site sequences were estimated using a logistic regression model, employing the glm function with binomial distribution, both implemented in the stats package for R (R Core team 2023). Bases at each position of spacer sequences, as well as arabinose concentration, were used as independent variables, and log odds of recombination were used as the dependent variable. Visual representation of the model was performed by generating a heatmap of its coefficients for different base-position combinations (FIG. 4D and FIG. 7D). For ease of interpretation, the coefficients were transformed to fold-change scale relative to on-target (loxP or loxSE3) recombination. Fold change was calculated by exponentiating positive coefficients and taking the negative reciprocal of the exponentiated coefficients for negative coefficients. When referencing the coefficients in the results section, the term “odds of recombination” or “chance of recombination” is used to describe the probability of a recombination event happening divided by the probability of the recombination event not happening.

Substrate-Linked Directed Evolution

New recombinases were evolved using the experimental principles as described in Lansing et al., 2020; Buchholz and Stewart 2001; Lansing et al., 2022; and in Karpinski et al., 2016 (FIG. 5B). To begin the evolution for increased activity on the target sites, a library of Cre mutants was generated by an error-prone PCR. Evolution for each site was done in parallel where the library was subjected to seven rounds of directed evolution and selected for improved activity on the given site. Positive selection pressure for activity on the novel spacer sequences loxSE1 (SEQ ID NO: 4), loxSE2 (SEQ ID NO: 5) or loxSE3 (SEQ ID NO: 6) was achieved through a modified method of substrate-linked directed evolution (FIG. 5B). Each cycle of evolution involves the diversification of the libraries through error prone PCR (MyTaq DNA Polymerase, Bioline) and selection of the variants for the desired activity on the presented target site.

The diversified libraries were first cloned into the pEVO containing the target site with the restriction sites of BsrGI and XbaI (purchased from NEB). The vector was then transformed into electrocompetent XL1-Blue E. coli and grown overnight in LB/arabinose to induce recombinase expression. To perform selection for recombination of the novel spacer sequence, the purified plasmid was digested with enzymes NdeI and AvrII (NEB) to linearize all non-recombined variants. A PCR was then performed with primers P5 and P6 to amplify only the clones that performed recombination (FIG. 5B). Recombination efficiency was monitored through the plasmid-based recombination assay (FIG. 1D)

Once activity on the given site was achieved, the libraries were enriched for active variants with three rounds of low induction and high-fidelity amplification (Herculase II Fusion DNA Polymerase, Agilent) resulting in increased activity of all libraries compared to Cre^{wt} (FIG. 5C). The DEQseq method (Schmitt et al., 2023) was then used for high-throughput evolution of the activity of individual recombinase variants from each library. The three libraries of active variants were barcoded via amplification, pooled together and then cloned into vectors for the DEQseq protocol. From the pool, around 5,000 variants were randomly selected and individual activity was quantified for all four target sites. Although only a portion of variants from each library were assayed, this method provides sequencing and activity of individual variants at multiple target sites, providing information not only on common mutational patterns and epistatic interactions but also a ranking system for activity-determining residue positions.

Further analysis of evolved recombinase variants was done with recombinases selected based on their activity across all four target sites and number of mismatches to Cre. Specific recombinases were defined as those with high on-target activity (the target site where the recombinase was selected for activity during evolution), low activity of the other three targets and high sequence identity to Cre^{wt} (SEQ ID NO: 8) (amino acid sequence identity greater than 95% to Cre^{wt}).

### Example 1: High-Throughput Analysis of Cre Activity on loxP-Spacer Library

To develop a comprehensive, high-throughput approach for profiling activity of Cre on loxP sites with matching mutant spacers, libraries of oligonucleotides were designed, each 120 nt long, encoding two identical mutant lox sites (FIG. 1B, Supplementary Figure S2C). Each oligonucleotide in the library was designed with the flanking 13 bp half-sites held constant as found in the wild-type loxP sequence (SEQ ID NO: 7) while mutations were systematically introduced to the core 8 bp spacer region resulting in a target site library of 6,000 distinct yet matching spacers. The library was then cloned to an expression vector encoding for Cre (wt sequence shown in SEQ ID NO: 8) (FIG. 1) and subsequently transformed to E. coli for recombinase expression (FIG. 2). To achieve precise quantification of recombination events with high sensitivity, high-throughput sequencing of the targets after PCR amplification was conducted, ensuring an average sequencing depth>900×coverage per target. Between biological replicates, the quantified recombination of library targets was consistent (median Pearson's R=0.98), demonstrating the reliability of the approach. These results indicate that the data are reproducible, thorough and at a comprehensive scale not previously assessed for recombinase/target activity. Quantification of Cre recombination at each target provided the means to investigate potential sequence preferences.

### Example 2: Systematic Characterization of Cre Recombinase Sequence Requirements for Functional lox Spacers

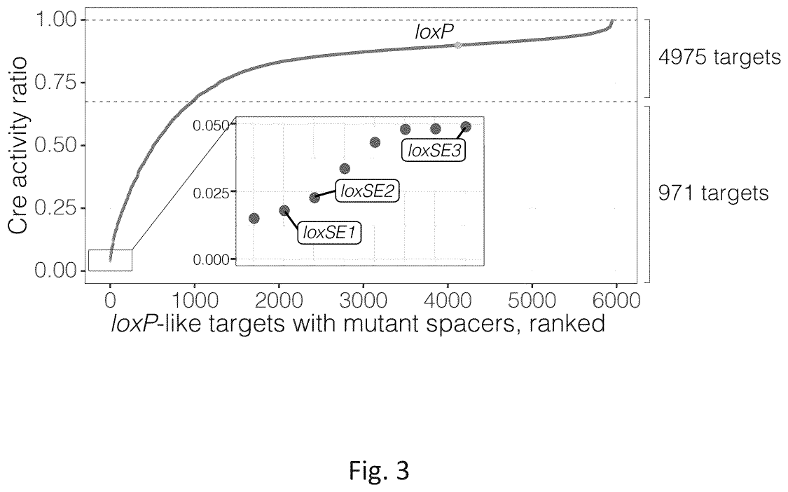

Consistent with the current literature (Hoess et al., 1986; Sheren et al., 2007, Lee et al., 1998), it was observed that 84% of the matching mutant spacers were efficiently recombined by Cre (FIG. 3). In order to compare the screen results to Crew/loxP efficiency, efficient recombination was defined as recombination within a range of +/−25% of the wild-type loxP recombination determined to be 87%. With this threshold, 16% of the target sites with mutant spacers were not efficiently recombined by Cre, with some sites showing less than 5% recombination. These findings underscore that efficient recombination is not guaranteed by spacer identity.

To investigate the results in more detail, it was first compared how number of mismatches from the loxP spacer sequence impacted the overall recombination (FIG. 4A). The screen results revealed a correlation between an increase in the number of mismatches from the canonical loxP sequence and a subsequent reduction in the potential for Cre recombinase activity. Specifically, target sites where both spacers had 7 or 8 mismatches from loxP exhibited a pronounced decrease in recombination efficiency. Of the targets with 7 or 8 mismatches, 86.7% and 100%, respectively, were inefficiently recombined by Cre (indicated by the shaded region, FIG. 4A) as stated above. Of the spacers with 6, 5, 4 and 3 mismatches, only 30%, 15.8%, 9.4%, and 7.6%, respectively, were inefficiently recombined. Nevertheless, the number of mismatches was not the only determinant of recombination efficiency. For instance, the target site with both spacers of sequence 5′-CAGTATTC-3′ (SEQ ID NO: 9) (bold font indicating mismatches from loxP), contains only 3 mismatches from the loxP spacer sequence (5′-ATGTATGC-3′) (SEQ ID NO: 10) and showed a recombination efficiency below 10%. On the other hand, spacer sequences containing 6 to 7 mutations from loxP (i.e., 5′-AATGTGTC-3′ (SEQ ID NO: 11) and 5′-TGAATTCG-3′ (SEQ ID NO: 12)), were efficiently recombined by Cre (88% and 78% recombined, respectively). These results suggest that, indeed, Cre reactions accommodate a wide range of spacer sequences for successful recombination. However, there is considerable variation in recombination efficiency across these sequences. This should be taken into consideration when selecting optimal target sites for reprogramming SSRs where parameters for target site selection focus on the half-sites, overlooking the specific DNA sequence of the spacer (Surendranath et al., 2010).

Cre recombination mismatch sensitivity highlights the sequence dependence of recombination, but it does not detail specific requirements for a functional spacer sequence. Therefore, the relationship between recombination and target sequence composition was further analyzed, as well as the context of base changes. To achieve this, we generated sequence logos for the top 10% recombined spacer sequences (n=595) and the bottom 10% recombined sequences (n=595) to visualize conserved patterns in each group (FIG. 4B, 4C). The logo of the top 10% of recombined spacer sequences demonstrates an enrichment of canonical bases A4′, G3 and C4, indicating a preference for the canonical loxP sequence in the flanking regions of the spacers for efficient recombination. Conversely, the logo of the bottom 10% of recombined spacer sequences shows an enrichment of base T3, suggesting that this base change negatively impacts efficient Cre recombination. Collectively, these findings reveal that base identity is an important determinant of efficient Cre-mediated recombination.

To classify the relationship between base substitutions at each position and recombination, a binomial generalized linear model (GLM) was employed, using recombination data from the comprehensive loxP spacer mutant library (FIG. 1G). The fold change in recombination rates resulting from single base changes was modeled to construct an activity profile for Cre. Analysis of the profile showed a preference for canonical loxP bases at positions A4′, G3 and C4 as illustrated in the logos (FIG. 4B, 4C). However, not all base changes at these positions have the same impact on recombination. For example, a base change from A-G at position 4′ or C-T at position 4 demonstrates a higher chance of recombination compared to other base changes at these sites (Stachowski et al., 2022, Abi-Ghanem et al., 2015; Lee et al., 1998). These variations can be attributed to the type of mismatch. Generally, base transitions (purine base substituted for another purine base, or pyrimidine base substituted for pyrimidine base) are more likely to be recombined by Cre than base transversions (purine base substituted for a pyrimidine base, or vice versa). Notably, a base transversion of G-T at position 3 presents the most significant reduction in successful recombination (−3.8; FIG. 4D) compared to any other base change. These results underscore the complex interplay between spacer sequence and efficient recombination. While sequence-based characteristics such as homology and mismatch count to loxP are generally indicative of efficient Cre recombination, the position and type of base change are also critical factors. These results suggest spacer sequence selectivity for efficient recombination in Cre.

### Example 3: Directed Evolution of Cre for Altered Spacer Sequence Preference

To test whether the restricted activity of Cre could be overcome, substrate-linked directed evolution (SLIDE) (Buchholz and Stewart, 2001) was performed to generated Cre mutants with increased activity on spacer sequences inefficiently recombined by Cre (FIG. 3). Three unique target sites, loxSE1, loxSE2 and loxSE3 (FIG. 5A), were selected from the mutant spacer screen based on their low recombination rates compared to wild-type loxP. Three major libraries were generated (LSE1, LSE2 and LSE3) by an error prone PCR of Cre followed by testing of the libraries on each of the sites (FIG. 5B, 5C). 11 cycles of SLIDE were performed in parallel for each library on the respective target sequences for increased activity (FIG. 5B). For all three libraries, increased band intensities representative of the recombined plasmid product were detected, indicating the enrichment of Cre variants with increased activity (FIG. 5C). To quantitatively investigate a large number of individual clones in the library, DNA Editing Quantification Sequencing (DEQSeq) was performed, which is a high-throughput Nanopore sequencing method that enables the characterization of thousands of DNA editing enzyme variants on multiple target sites (Schmitt et al., 2023). A total of about 5,000 random variants were selected from the three libraries and quantified recombination activity of each of the variants on all four sites (loxP, loxSE1, loxSE2 and loxSE3) at an average sequencing depth of>1000× reads per variant. Experiments using conventional agarose gel quantifications confirmed the validity of the approach.

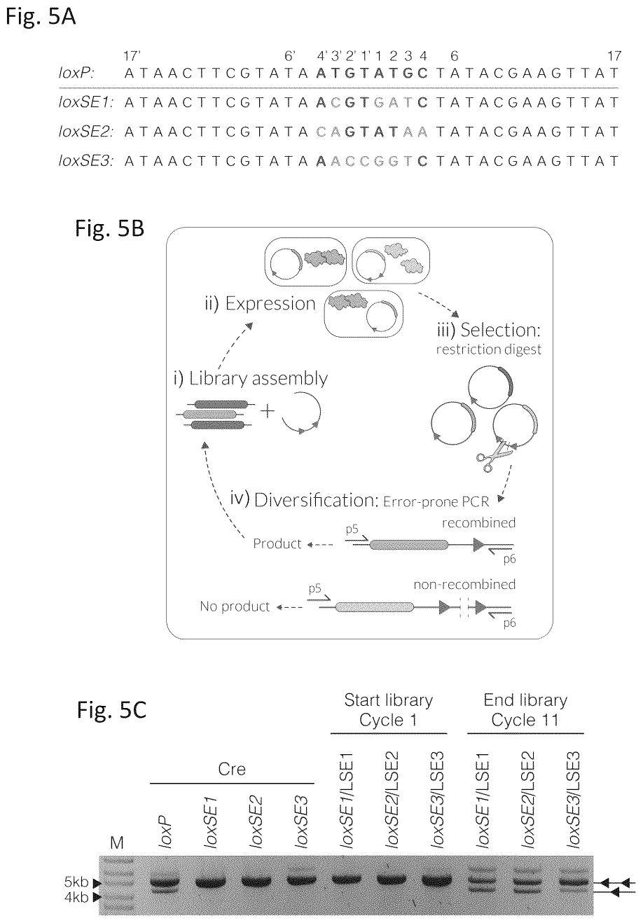

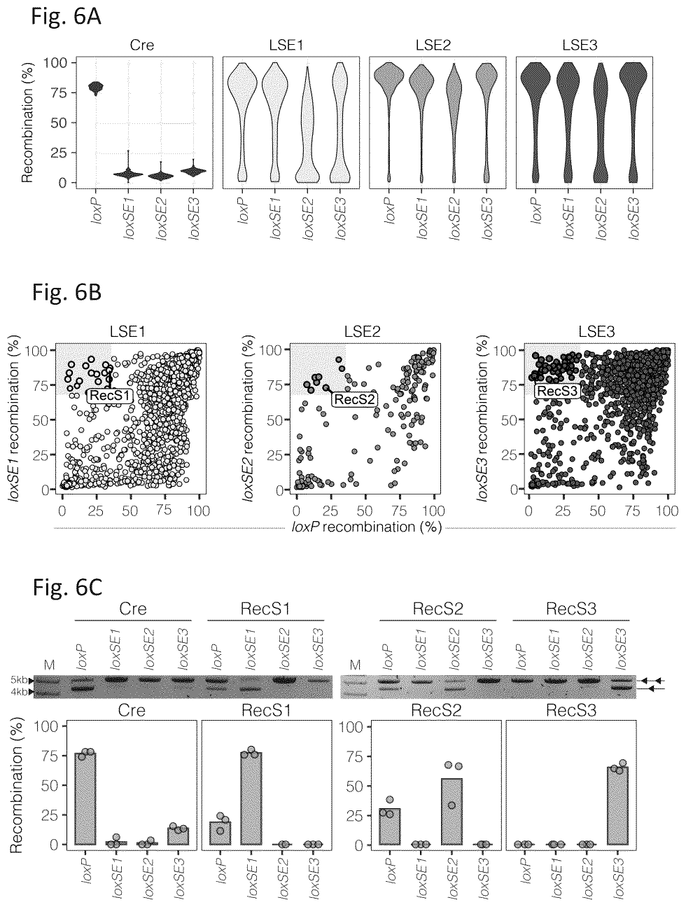

By comparing Cre and the library variants distribution of activities for loxP, loxSE1, loxSE2 and loxSE3, a clear increase in activity of many of the recombinase variants on their respective evolved target site was seen across all libraries while maintaining high activity on the parental loxP wild-type spacer (FIG. 6A). Because the selection criteria for evolution was activity and not specificity, many variants emerged with the capacity of efficiently recombining all four spacer sequences.

Surprisingly, although many recombinase variants evolved an overall increase of activity on all four target sites, <1% of the recombinase variants became unproductive on the parental wild-type loxP spacer (FIG. 6B) and “switched” their activity towards the new spacer. This result suggested that clones had emerged that had lost their ability to efficiently recombine the wild-type loxP spacer, while gaining activity on the mutant spacer. Of the evolved spacer-specific recombinases, three examples were selected (RecS1 (SEQ ID NO: 13), RecS2 (SEQ ID NO: 14) and RecS3 (SEQ ID NO: 15); FIG. 6B) to further compare their activity across all targets. Testing the three recombinases in a plasmid-based assay indeed revealed that these enzymes preferentially recombine their selected spacer sequences (FIG. 6C), indicating that Cre variants can be generated with spacer sequence selectivity. Of the three variants, RecS3 was the most interesting due to the drastic shift in spacer activity to recombine only the loxSE3 target without prominent activity on any of the other sites (FIG. 6C). These results establish the programmability of Cre recombinase to selectively recombine sequences with different spacers.

### Example 4: High-Throughput Analysis of RecS3 Activity on loxP-Spacer Library

To comprehensively determine the spacer requirements for functional RecS3 recombination (selected for target site loxSE3; FIG. 5A) and how those requirements differ from Cre, the same target recombination assay as used with Cre by quantifying RecS3 activity on the 6,000 loxP-like spacer variants was applied (FIG. 7A). Plotting the activities of RecS3 compared to Cre on all target sites in the library (FIG. 7A) shows that Cre recombines a wider range of spacer sequences more efficiently than RecS3 (indicated by the density of target sites in the bottom right of the graph; FIG. 7A). Nevertheless, RecS3 efficiently recombines numerous spacer sequences, including some that are inefficiently recombined by Cre (upper left quadrant of the graph; FIG. 7A). These results provide a direct comparison of spacer sequence selectivity of RecS3 and Cre.

To visualize the selective recombination preferences of RecS3, the conserved characteristics leading to efficient recombination were plotted as logos of the top 10% sequences with the highest activity and the bottom 10% sequences with the lowest activity (FIG. 7B, 7C). Comparing these logos to the Cre logos (FIG. 4B, 4C), a clear difference in spacer sequence preference can be deduced for the two recombinases. In the context of RecS3-mediated recombination, the analysis of base frequencies among the top recombined sequences revealed a pronounced selectivity, with a base frequency exceeding 40%, for loxSE3 at positions A4′, G2, T3, and C4. Intriguingly, an additional observation underscores a notable preference for a non-loxSE3 base, T2′. Conversely, examination of base frequencies in the least recombined sequences highlighted a heightened occurrence of bases G2′, T1′,A1, and T2, all of which are integral components of the wild-type loxP sequence. This notable prevalence within the inefficiently recombined sequences of specific bases in the wild-type loxP sequence may explain the observed reduction in RecS3 activity for loxP. Collectively, these findings emphasize the preference of RecS3 towards the loxSE3 site.

The RecS3 spacer specificity was further evaluated by constructing a profile with recombination rates from all spacer substrates (same application as done with Cre, see GLM model formulation in Methods for calculation, FIG. 7D). As observed in the spacer profile of Cre, RecS3 was more permissive to base transitions than base transversions, most notably, the base transversions of C-G2′, decreasing the odds of recombination by 3.5-fold, and the base transversion of A-C4′, decreasing the odds of recombination by 4.4-fold. Additionally, RecS3 displayed a high sensitivity to all base changes at G2. Comparing the specificity profiles of Cre and RecS3, both recombinases were similarly sensitive to base changes in positions 3′ and 3. In contrast, RecS3 exhibited heightened sensitivity to alterations in positions 4′ and 2, accompanied by a diminished sensitivity to base changes at position 4. A comparative analysis of the two profiles underscored the increased specificity of RecS3, emphasizing the critical roles of bases G2 and T3 in RecS3 recombination, observations not evident in Cre. These findings emphasize the unique and intrinsic specificity shift characterizing RecS3, setting it apart from Cre.

### Example 5: Molecular Dissection of Specificity Switch

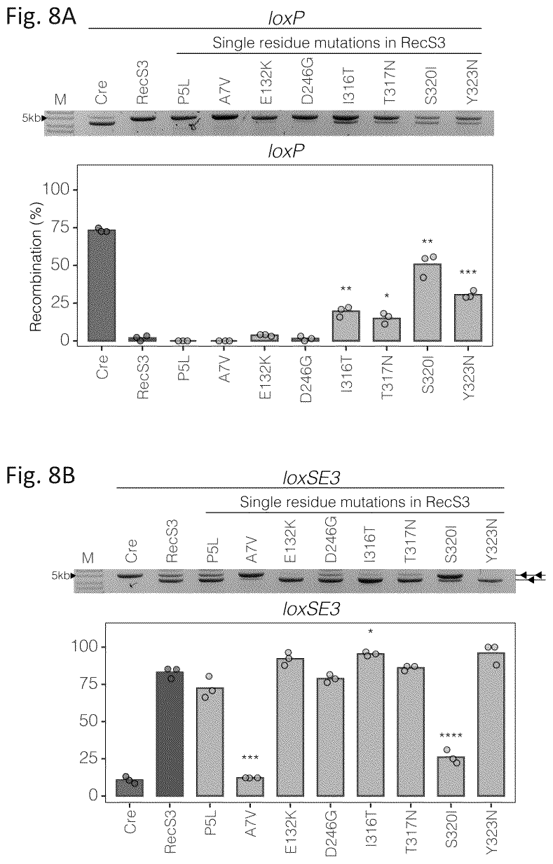

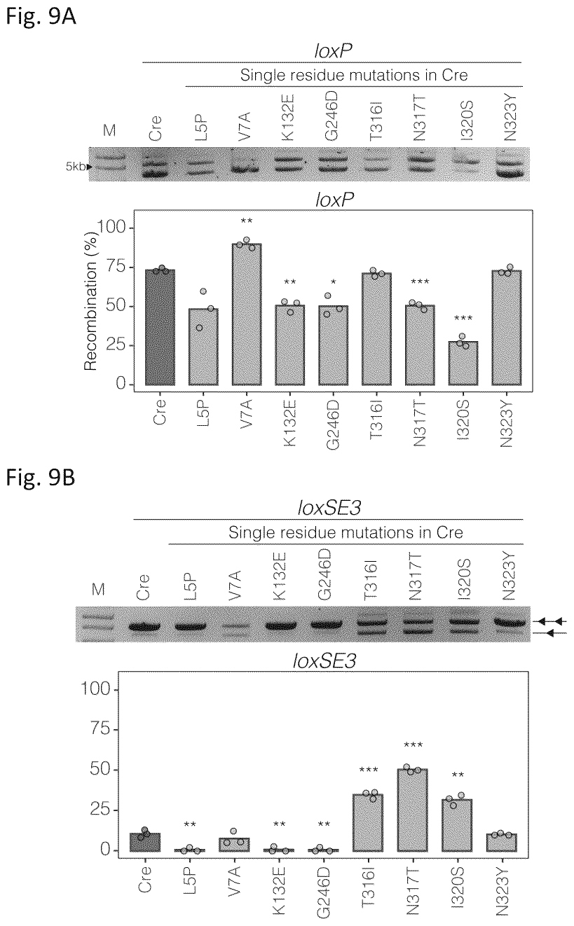

RecS3 (SEQ ID NO: 15) contains eight mutations compared to Cre^{wt}: L5P, V7A, K132E, G246D, T316I, N317T, I320S, and N323Y. To assess the specific impact of individual mutations on the overall activity of loxP and loxSE3, eight RecS3 mutants were generated, where each contained a single mutation back to the Cre^{wt} residue. Among the RecS3 mutants examined, half of the single residue reversion mutants to Cre^{wt} (RecS3: P5L, E132K, D246G, and T317N) individually demonstrated no significant alteration in the overall activity concerning loxSE3 and loxP (FIG. 8A, 8B). This is most likely due to the passive nature of these mutations acquired during evolution or that these residue changes are acting in concert with the other mutations. Interestingly, the mutants RecS3^{A}7V and RecS3^{S320I} showed a significant loss of activity on loxSE3 (FIG. 8B). Moreover, RecS3^{S320I} significantly increased the activity on loxP, implying that S320I plays a key role in the selectivity for different spacer sequences. In contrast, RecS3^{A}7V showed diminished activity on both loxP and loxSE3, suggesting a role of this mutation for overall recombination function, possibly related to the stability of the recombinase (Guillén-Pingarrón et al., 2022). The individual RecS3 mutations in Cre were also analyzed (FIG. 9A, 9B).

### Example 6: Molecular Modelling and Dynamics Simulations: Analysis of the Molecular Basis for Activity and Target Selectivity

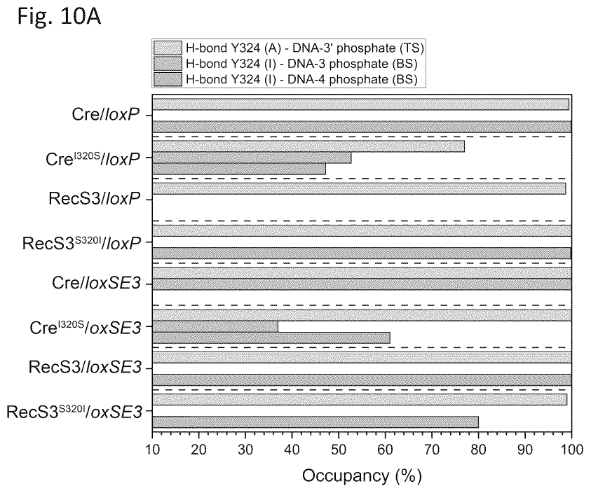

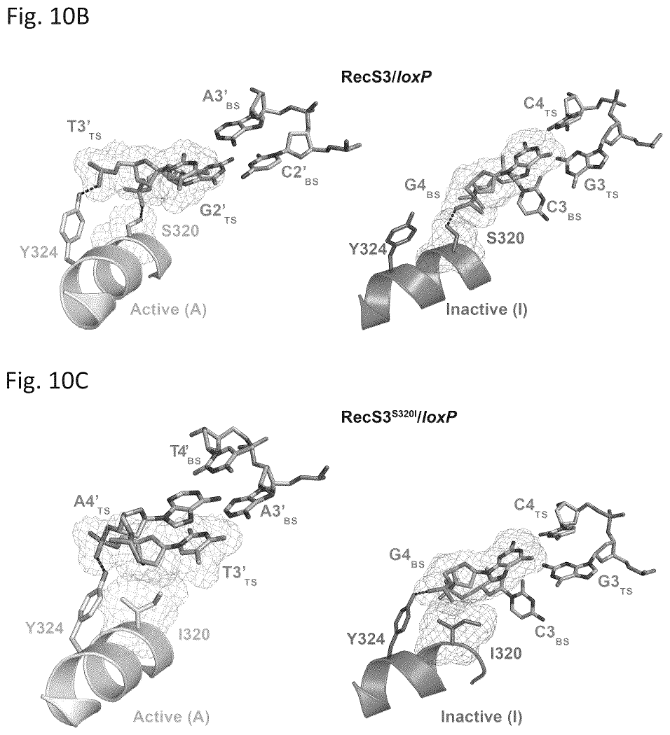

3D molecular models of the complexes of Cre and RecS3 and the most prominent mutation at position 320 with respect to loxP and loxSE3 were generated: Cre^{I320S}/loxP, RecS3/loxP, RecS3^{S320I}/loxP, Cre^{wt}/loxSE3, Cre^{I320S}/loxSE3, RecS3/loxSE3 and RecS3^{S320I}/loxSE3 (FIG. 10A). These models were based on the high resolution synaptic Cre/loxP complex PDB ID 3C29 (see Methods). In this structure, the Cre active (A) monomer exhibits the catalytic residues positioned for top strand (TS) cleavage like in other crystallographic structures available in the Protein Data Bank (Ennifar et al., 2003). For the modelling and analysis, only the dimer unit was considered (i.e. two Cre monomers—one active and the other inactive—in complex with one molecule of the corresponding DNA target site (i.e. loxP or loxSE3)).

MD simulations of the investigated Cre mutants and the wild-type system and the subsequent comparative H-bond analysis at the catalytic site revealed clear variations in DNA recognition by the catalytic residue Y324 (FIG. 10A). In the RecS3/loxP complex, Y324 of the inactive (I) monomer (Y324(I)) exhibits a striking shift with respect to the positioning observed in Cre_{wt} (FIG. 10A, 10B) resulting in the loss of the interaction with the DNA phosphate backbone (p) at base G4 of the bottom strand (G4(BS)). Furthermore, no interaction is observed between the catalysis-relevant residue K201(I) and the sugar backbone of base T5(TS). In the context of the active (A) monomer, no interactions of RecS3 with pA5′(TS) and pA4′(TS) are detected. As a consequence, H289(A) is not optimally positioned to facilitate recombination compared to Cre. The observed configurational shift in Y324(I) together with the loss of interactions of critical residues for catalysis such as K201(I) and H289(A) could explain the diminished activity of RecS3 towards loxP. Conversely, in the context of the loxSE3 target site, residues Y324, K201 and H289 of RecS3 were found appropriately positioned in both the active and inactive monomers (FIG. 10A). Notably, the recognition of loxSE3 by Cre_{wt} was predicted with Y324(I) shifted towards pA3(BS) instead of contacting pG4(BS). Interestingly, Y283(I) was observed establishing contacts with pG4(BS). Moreover, no interactions with pA5′(BS) were observed, and the interactions of K201(A) with A4′(TS) as well as I320 and N317 with pC2′(TS) were not detected. These differences in the recognition pattern of loxSE3 by Cre^{wt} compared to RecS3 could explain why Cre^{wt} exhibits inefficiency as recombinase for loxSE3. The observed displacement of Y324(I) suggests its positioning for DNA cleavage, also in the inactive monomer. Recombination occurs through a stepwise cleavage process with synapsis formation (i.e. active tetrameric complex) involving two dimer complexes with cleavage preference for either the BS or TS (i.e. formation of a BS-BS or TS-TS synapsis resulting in a BS or TS first cleavage, respectively). Recent work analyzing pre-synaptic Cre/loxP dimers has suggested that the formation of a BS-TS complex would not be compatible with effective recombination activity (Stachowski et al., 2022; Martin et al., 2002).

Next, the molecular basis accounting for the experimentally observed DNA target specificity was investigated, in particular the relevance of the residue at position 320 neighboring the catalytic Y324. Theoretical models predicted a preference for isoleucine at position 320 when the target site is loxP. This preference arises from van der Waals contacts between the side chain of I320(A) and the sugar backbone and base T3′(TS), as observed in Cre^{wt} and RecS3^{S320I} (FIG. 10C). These interactions diminish when isoleucine is substituted by serine. Nevertheless, the recombination activity of RecS3^{S320I} does not reach the levels of Cre^{wt}, which may arise from the lack of interactions of I320 with pG2′(TS) and of K201(A) with A4′(TS). Of particular interest is the behavior of Y324(I) of Cre^{I320S}, which interacts with pC3(BS) and pG4(BS) of loxP (53% and 47% of the MD simulation time, respectively; FIG. 10A) and with pA3(BS) and pG4(BS) of loxSE3(37% and 61% of the MD simulation time, respectively). These interactions in both target sites might further compromise the catalytic activity of these recombinases. Furthermore, residue W315(A), which also supports recombination, does not recognize pT3′(TS) of loxP and pA3′(TS) of loxSE3. For RecS3, residue S320(A) establishes an H-bond with pC2′(TS) in loxSE3, as it is observed in Cre^{I320S}. In contrast, such interactions are not observed in the RecS3^{S320I}/loxSE3 complex.

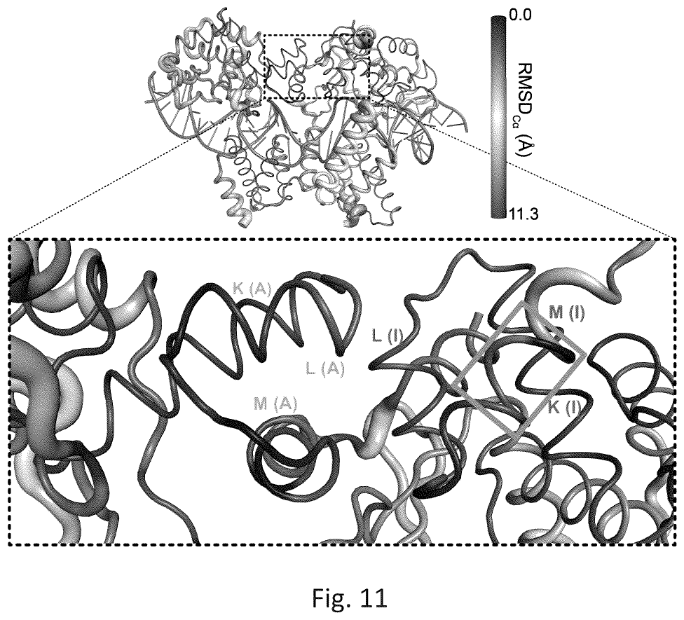

The fact that several catalytic residues (i.e. H289, W315, Y324) and some of the mutations introduced in RecS3 with respect to Cre^{wt} (i.e. 1316T, T317N, S320I, Y323N) are contained in the helices K, L and M of the recombinase, and that the recognition of these regions by the DNA could perhaps account for the predicted displacement of Y324 in the studied systems, led to an investigation of potential conformational alterations in these regions. Calculated RMSD_{Cα} values from average MD structures showed negligible conformational changes. However, residues Y324 (I) and 1325 (I) of RecS3 exhibited the highest RMSD_{Cα} values when complexed to loxP, indicating their displacement in comparison to Cre/loxP (FIG. 11).

The high-throughput approach described herein allows for profiling recombinase activity of target libraries with matching mutant spacers. Because each target in the library is predetermined and not randomized, it is possible to systematically isolate and compare sequence-based features of the spacer region to the overall effect on recombination, all while maintaining symmetry of the target sites. Although the library used only covers a portion of all possible combinations in the spacer sequence (6,000 of the 65,536 (48) possibilities), a solid basis was built to profile the influence of the spacer sequence on recombination activity. The data generated can be useful to train deep learning models with the aim of predicting spacer sequence preferences for SSRs (Schmitt et al., 2022). Screening the activity of Cre revealed that spacer homology and sequence identity to loxP generally indicate efficient recombination, although certain base changes can overcome this rule, suggesting that Cre recombination is more dependent on spacer sequence than previously thought (Lee et al., 2003; Sheren et al., 2007; Lee and Saito, 1998).

To explore the dependency of the spacer sequence for efficient recombination, spacers were selected that are inefficiently recombined by Cre. Directed evolution was applied to increase activity on these sites. Surprisingly, only eleven cycles of evolution achieved solely via positive selection, recombinase variants were generated with an overall heightened activity. In addition, variants were uncovered that exhibited a switch in specificity. Certain recombinase variants demonstrated a gain of activity on the new spacer sequence, while concurrently losing activity on the parental loxP site, even though no negative selection against loxP was applied. From the variants with altered spacer specificity, RecS3 was further characterized. By comprehensively screening the activity of RecS3 using the spacer library, it was found that RecS3 has a different set of spacer requirements compared to Cre, which might be related to variations on DNA-protein interactions and differences in the intrinsic flexibility provided by the spacer sequence, making it the first recombinase to be successfully engineered for spacer specificity. This represents a significant finding by providing the opportunity to fine tune activity on a target site uniquely based on the spacer sequence.

The present invention provides a platform for modifying the spacer specificity of DNA recombining enzymes, adding an additional layer of specificity, and increasing the potential for engineering of DNA recombining enzymes in particular for therapeutic applications.

### Cited Non-Patent Literature

Abi-Ghanem, J, Chusainow, J, Karimova, M, Spiegel, C, Hofmann-Sieber, H, Hauber, J, Buchholz, F and Pisabarro, M T (2013). Engineering of a target site-specific recombinase by a com-bined evolution-and structure-guided approach. Nucleic Acids Res., 41, 2394-2403.
Abi-Ghanem, J, Samsonov, S A and Pisabarro, M T (2015). Insights into the preferential order of strand exchange in the Cre/loxP recombinase system: impact of the DNA spacer flanking se-quence and flexibility. J. Comput.-Aided Mol. Des., 29, 271-282.
Abremski, K, Hoess, R and Sternberg N (1983). Studies on the properties of P1 site-specific re-combination: Evidence for topologically unlinked products following recombination. Cell, 32, 1301-1311
Anzalone A V, Gao X D, Podracky C J, Nelson A T, Koblan L W, Raguram A, Levy J M, Mercer J A M, Liu D R. Programmable deletion, replacement, integration and inversion of large DNA sequences with twin prime editing. Nat Biotechnol. 2022 May;40(5):731-740.
Aznauryan E, Yermanos A, Kinzina E, Devaux A, Kapetanovic E, Milanova D, Church G M, Reddy S T. Discovery and validation of human genomic safe harbour sites for gene and cell therapies. Cell Reports Methods 2022 January:2(1):100154.
Buchholz, F, and Stewart, A F (2001). Alteration of Cre recombinase site specificity by substrate-linked protein evolution. Nat Biotechnol 19, 1047-1052.
Buchholz, F and Hauber, J (2011). In vitro evolution and analysis of HIV-1 LTR-specific recom-binases. Methods, 53, 102-109.
Durrant MG, Fanton A, Tycko J, Hinks M, Chandrasekaran S S, Perry N T, Schaepe J, Du P P, Lotfy P,
Bassik M C, Bintu L, Bhatt A S, Hsu P D. Systematic discovery of recombinases for efficient integration of large DNA sequences into the human genome. Nat Biotechnol. 2023 April;41(4):488-499. doi: 10.1038/s41587-022-01494-w. Epub 2022 Oct. 10. PMID: 36217031; PMCID: PMC10083194.
Duyne, G D V (2001). A Structural View of Cre-loxP Site-Specific Recombination. Annu. Rev. Biophys. Biomol. Struct., 30, 87-104.
Engler, C, Kandzia, R and Marillonnet, S (2008). A One Pot, One Step, Precision Cloning Method with High Throughput Capability. PLoS ONE, 3, e3647.
Ennifar, E, Meyer, J E W, Buchholz, F, Stewart, A F and Suck, D (2003). Crystal structure of a wild-type Cre recombinase-lox P synapse reveals a novel spacer conformation suggesting an alternative mechanism for DNA cleavage activation. Nucleic Acids Res, 31, 5449-5460.
Gelato, K A, Martin, S S and Baldwin, E P (2005). Reversed DNA Strand Cleavage Speci-ficity in Initiation of Cre-LoxP Recombination Induced by the His289Ala Active-site Substitution. J. Mol. Biol., 354, 233-245.
Ghosh, K and Duyne, G D V. Cre-loxP Synaptic structure. 10.2210/pdb3c29/pdb.
Gopaul, DN, Guo, F and Duyne, GDV (1998). Structure of the Holliday junction intermediate in Cre-loxP site-specific recombination. EMBO J., 17, 4175-4187.
Grindley, NDF (1997). Site-specific recombination: Synapsis and strand exchange revealed. Curr. Biol., 7, R608-R612.
Grindley, N D F, Whiteson, K L and Rice, P A (2006). Mechanisms of site-specific recombination. Annu Rev Biochem, 75, 567-605.
Guillén-Pingarrón, C, Guillem-Gloria, P M, Soni, A, Ruiz-Gómez, G, Augsburg, M, Buchholz, F, Anselmi, M and Pisabarro, M T (2022). Conformational dynamics promotes disordered regions from function-dispensable to essential in evolved site-specific DNA recombinases. Comput Struct Biotechnology J, 20, 989-1001.
Guo, F, Gopaul, D N and Duyne, G D V (1997). Structure of Cre recombinase complexed with DNA in a site-specific recombination synapse. Nature, 389, 40-46.
Guo, F, Gopaul, D N and Duyne, G D V (1999). Asymmetric DNA bending in the Cre-loxP site-specific recombination synapse. Proc. Natl. Acad. Sci., 96, 7143-7148.
Heddi, B, Oguey, C, Lavelle, C, Foloppe, N and Hartmann, B (2010). Intrinsic flexibility of B-DNA: the experimental TRX scale. Nucleic Acids Res, 38, 1034-1047.
Hoersten, J, Ruiz-Gómez, G, Lansing, F, Rojo-Romanos, T, Schmitt, L T, Sonntag, J, Pisabar-ro, M T and Buchholz, F (2021). Pairing of single mutations yields obligate Cre-type site-specific recombinases. Nucleic Acids Res, 50(2), 1174-1186.
Hoess, R H, Ziese, M and Sternberg, N (1982). P1 site-specific recombination: nucleotide se-quence of the recombining sites. Proc. Natl. Acad. Sci., 79, 3398-3402.
Hoess, R H, Wierzbicki, A and Abremski, K (1986). The role of the loxP spacer region in PI site-specific recombination. Nucleic Acids Res, 14, 2287-2300.
Humphrey, W, Dalke, A and Schulten, K (1996). VMD: Visual molecular dynamics. J Mol Graphics, 14, 33-38.
Jelicic, M. et al. (2023). Discovery and characterization of novel Cre-type tyrosine site-specific recombinases for advanced genome engineering. Nucleic Acids Res. 51 (10), 5285-5297.
Jung, U-J, Park, S, Lee, G, Shin, H-J and Kwon, M-H (2007). Analysis of spacer regions derived from intramolecular recombination between heterologous loxP sites. Biochem Bioph Res Co, 363, 183-189.
Karst S M, Ziels R M, Kirkegaard R H, Sørensen E A, McDonald D, Zhu Q, Knight R, Albertsen M (2021). High-accuracy long-read amplicon sequences using unique molecular identifiers with nanopore or PacBio sequencing. Nat Methods 18:165-169; available from: https://doi.org/10.1038%2Fs41592-020-01041-y.
Karpinski, J, Hauber, I, Chemnitz, J, Schäfer, C, Paszkowski-Rogacz, M, Chakraborty, D, Be-schorner, N, Hofmann-Sieber, H, Lange, U C, Grundhoff, A, et al. (2016). Directed evolution of a recombinase that excises the provirus of most HIV-1 primary isolates with high specificity. Nat Biotechnol, 34, 401-409.
Kechin, A, Boyarskikh, U, Kel, A and Filipenko, M (2017). cutPrimers: A New Tool for Accurate Cutting of Primers from Reads of Targeted Next Generation Sequencing. J. Comput. Biol., 24, 1138-1143.
Lansing, F, Paszkowski-Rogacz, M, Schmitt, L T, Schneider, P M, Romanos, T R, Sonntag, J and Buchholz, F (2020). A heterodimer of evolved designer-recombinases precisely excises a human genomic DNA locus. Nucleic Acids Res, 48, 472-485.
Lansing, F, Mukhametzyanova, L, Rojo-Romanos, T, Iwasawa, K, Kimura, M, Paszkowski-Rogacz, M, Karpinski, J, Grass, T, Sonntag, J, Schneider, P M, et al. (2022). Correction of a Factor VIII genomic inversion with designer-recombinases. Nat Commun, 13, 422.
Lavery, R, Moakher, M, Maddocks, J H, Petkeviciute, D and Zakrzewska, K (2009) Con-formational analysis of nucleic acids revisited: Curves+. Nucleic Acids Res., 37, 5917-5929.
Lee, G and Saito, I (1998). Role of nucleotide sequences of loxP spacer region in Cre-mediated recombination. Gene, 216, 55-65.
Lee, L and Sadowski, P D (2003). Sequence of the loxP Site Determines the Order of Strand Ex-change by the Cre Recombinase. J Mol Biol, 326, 397-412.
Luetke, K H and Sadowski, P D (1995). The Role of DNA Bending in F1p-mediated Site-specific Recombination. J. Mol. Biol., 251, 493-506.
Martin, S S, Pulido, E, Chu, V C, Lechner, T S and Baldwin, E P (2002). The Order of Strand Ex-changes in Cre-LoxP Recombination and its Basis Suggested by the Crystal Structure of a Cre-LoxP Holliday Junction Complex. J Mol Biol, 319, 107-127.
Meinke, G, Bohm, A, Hauber, J, Pisabarro, M T and Buchholz, F (2016). Cre Recombinase and Other Tyrosine Recombinases. Chem. Rev., 116, 12785-12820.
Missirlis, P I, Smailus, D E and Holt, R A (2006). A high-throughput screen identifying sequence and promiscuity characteristics of the loxP spacer region in Cre-mediated recombination. BMC Genom., 7, 73.
Pellenz S, Phelps M, Tang W, Hovde B T, Sinit R B, Fu W, Li H, Chen E, Monnat R J jr. New Human Chromosomal Sites with “Safe Harbor” Potential for Targeted Transgene Insertion. Hum Gene Ther. 2019 Jul. 1; 30(7): 814-828.
R Core Team (2023). R: A language and environment for statistical computing. Founda-tion for Statistical Computing, Vienna, Austria. (2023).
Rognes T, Flouri T, Nichols B, Quince C, Mahé, F. VSEARCH: a versatile open source tool for metagenomics. PeerJ 2016, 1-22.
Rojo-Romanos, T, Karpinski, J, Millen, S, Beschorner, N, Simon, F, Paszkowski-Rogacz, M, Lansing, F, Schneider, P M, Sonntag, J, Hauber, J, et al. (2023). Precise exci-sion of HTLV-1 provirus with a designer-recombinase. Mol. Ther., 31, 2266-2285.
Sarkar, I, Hauber, I, Hauber, J and Buchholz, F (2007). HIV-1 Proviral DNA Excision Using an Evolved Recombinase. Science, 316, 1912-1915.
Schmitt, L T, Paszkowski-Rogacz, M, Jug, F and Buchholz, F (2022). Prediction of designer-recombinases for DNA editing with generative deep learning. Nat. Commun., 13, 7966.
Schmitt, L T, Schneider, A, Posorski, J, Lansing, F, Jelicic, M, Jain, M, Sayed, S, Buchholz, F and Sürün, D (2023). Quantification of evolved DNA-editing enzymes at scale with DEQSeq. Genome Biol., 24, 254.
Sheren, J, Langer, S J. and Leinwand, L A (2007). A randomized library approach to identifying unctional lox site domains for the Cre recombinase. Nucleic Acids Res, 35, 5464-5473.
Stachowski, K, Norris, A S, Potter, D, Wysocki, V H and Foster, M P (2022). Mechanisms of Cre recombinase synaptic complex assembly and activation illuminated by Cryo-EM. Nucleic Acids Res, 50, 1753-1769.
Sternberg, N, Hamilton, D and Hoess, R (1981). Bacteriophage P1 site-specific recombination II. Recombination between loxP and the bacterial chromosome. J Mol Biol, 150, 487-507.
Surendranath, V, Chusainow, J, Hauber, J, Buchholz, F and Habermann, B H (2010). SeLOX—a locus of recombination site search tool for the detection and directed evolu-tion of site-specific recombination systems. Nucleic Acids Res., 38, W293-W298.
Wagih, O (2017). ggseqlogo: a versatile R package for drawing sequence logos. Bioinfor-matics, 33, 3645-3647.
Yarnall M T N, Ioannidi E I, Schmitt-Ulms C, Krajeski R N, Lim J, Villiger L, Zhou W, Jiang K, Garushyants S K, Roberts N, Zhang L, Vakulskas C A, Walker J A 2nd, Kadina A P, Zepeda A E, Holden K, Ma H, Xie J, Gao G, Foquet L, Bial G, Donnelly S K, Miyata Y, Radiloff D R, Henderson J M, Ujita A, Abudayyeh O O, Gootenberg J S. Drag-and-drop genome insertion of large sequences without double-strand DNA cleavage using CRISPR-directed integrases. Nat Biotechnol. 2023 April;41(4):500-512. doi: 10.1038/s41587-022-01527-4. Epub 2022 Nov. 24. PMID: 36424489; PMCID: PMC10257351.
Zhu F, Gamboa M, Farruggio A P, Hippenmeyer S, Tasic B, Schule B, Chen-Tsai Y, Calos M P. DICE, an efficient system for iterative genomic editing in human pluripotent stem cells. Nucleic Acids Res. 2014 March;42(5):e34. doi: 10.1093/nar/gkt1290. Epub 2013 Dec. 4. PMID:24304893; PMCID: PMC3950688.
Zurek P. J., Knyphausen P., Neufeld K., Pushpanath A., Hollfelder F. (2020). UMI-linked consensus sequencing enables phylogenetic analysis of directed evolution. Nature Communications 11; available from: https://doi.org/10.1038/s41467-020-19687-9.

1. Method for generating a spacer-specific DNA recombining enzyme (DRE), the method comprising the steps of:
a) providing a 1. library of expression vectors encoding a plurality of 1. variants of a DRE (vDRE), wherein the amino acid sequences of the 1. vDREs comprise one or more amino acid modifications in comparison to a non-variant DRE (nvDRE) from which the 1. vDREs are derived, wherein the nvDRE binds to a first target site comprising in 5′ to 3′ direction a half-site A (HSA), a spacer (Spacer) and a half-site B (HSB), wherein each expression vector comprises:
(i) a first region comprising a nucleotide sequence encoding one of the 1. vDREs from among the plurality of 1. vDREs operably linked to an expression control sequence, and
(ii) a second region comprising a nucleotide sequence comprising in 5′ to 3′ direction a first target site, an insert nucleotide sequence (INS) of a length at least 1 nucleotide and a second target site, wherein each of the first and second target site comprises the HSA, a 1. variant spacer (vSpacer) and the HSB, wherein the 1. vSpacer differs by at least one nucleotide from the Spacer;
b) introducing the library of expression vectors into host cells;
c) expressing the plurality of 1. vDREs in the host cells;
d) optionally isolating DNA from the host cells; and
e) determining whether the 1. vDRE shows activity on the target sites of at least one expression vector;
optionally repeating steps a) to e) until a 1. vDRE is produced that shows activity on the target sites of at least one expression vector.

2. The method according to claim 1, further comprising the steps of:
f) providing 1+n. libraries of expression vectors encoding a plurality of 1+n. variants of the vDREs (1+n. vDREs), wherein the 1+n. vDREs comprise one or more amino acid modifications in comparison to the n. vDREs, wherein each expression vector comprises:
(i) a first region comprising a nucleotide sequence encoding one of the 1+n. vDREs from among the plurality of the 1+n. vDREs operably linked to an expression control sequence, and
(ii) a second region comprising a nucleotide sequence comprising a first and a second target site 5′ and 3′, respectively, of an insert nucleotide sequence (INS) of a length at least 1 nucleotide, wherein each of the first and second target site comprises the HSA, a 1+n. variant spacer (1+n. vSpacer) and the HSB, wherein the 1+n. vSpacer differs by at least one nucleotide from the 1. vSpacer;
g) introducing the 1+n. libraries of expression vectors into host cells;
h) expressing the plurality of 1+n. vDREs in the host cells;
i) isolating DNA from the host cells; and
j) determining whether the 1+n. vDREs show activity on the target sites of at least one expression vector, wherein n≥1.

3. The method of claim 2, further comprising repeating steps f) to j) until a 1+n. vDRE is produced that excises the INS.

4. The method of claim 2, wherein n is increased by 1 once a 1+n. vDRE is produced that excises the INS.

5. The method according to claim 1,
wherein the Spacer and the n or 1+n. variant Spacer each have a length of between 6 and 10 nucleotides, preferably 8 nucleotides; and/or
wherein the HSA and the HSB each have a length of between 11 and 15 nucleotides, preferably 13 nucleotides.

6. The method according to claim 1, wherein the nvDRE is a naturally occurring DRE or a variant thereof that binds to a first and second half site different from the first and second half site bound by the naturally occurring DRE, preferably a tyrosine recombinase or a large serine recombinase,
preferably a tyrosine recombinase selected from the group consisting of Cre-, Dre-, VCre-, SCre-, Vika-, lambda-Int-, F1p-, R-, Kw-, Kd-, B2-, B3-, Nigri- or Panto-recombinases, or
preferably a large serine recombinase selected from the group consisting of A118, TP901, φRV1, Bxb1, φC31, R4, Wβ, Tnpx, Cp36, Dn29, Kp03, Nm60, Pa01, Si74.

7. The method according to claim 1, wherein step e) or j) of determining whether the 1. vDRE shows activity on the target sites of at least one expression vector comprises:
i) performing PCR on the host cell of c) or h) or the isolated DNA of step d) or i) with a first primer specifically hybridizing 5′ of or partially overlapping or fully overlapping with the first target site, and a second primer specifically hybridizing 3′ of or partially overlapping or fully overlapping with the second target site, and optionally sequencing of the PCR product; or ii) restriction digestion of the expression vector with one or more restriction enzymes that cleave the INS.

8. The method according to claim 1, further comprising the step of removing inactive variants of the 1. or 1+n. vDREs from the library of expression vectors.

9. A vDRE obtainable by the method according to claim 1, wherein the amino acid sequence of the vDRE differs in at least one amino acid from the nvDRE, preferably wherein the vDRE comprises or consists of an amino acid sequence having at least 85% identity to any one of SEQ ID NOs: 13, 14, or 15.

10. A nucleic acid or group of nucleic acids encoding a vDRE according to claim 9.

11. An expression vector comprising a nucleic acid or group of nucleic acids according to claim 10.

12. A system for (i) integrating a donor nucleic acid into a target nucleic acid, or (ii) exchanging a [a] nucleic acid sequence of interest in the genome of a subject or cell for a different nucleic acid sequence, the system comprising
a polypeptide comprising a vDRE according to claim 9; and
either (i) a donor nucleic acid to be inserted into the target nucleic acid, or (ii) a nucleic acid sequence differing from the nucleic acid sequence to be exchanged.

13. A pharmaceutical composition comprising the vDRE of claim 9, and optionally a pharmaceutically acceptable carrier.

14. A method for (i) integrating a nucleic acid sequence of interest into the genome of a subject or cell, or (ii) for exchanging a nucleic acid sequence of interest in the genome of a subject or cell for a different nucleic acid sequence, wherein the cell does not include cells of the human germ line, the method comprising introducing into the cell the vDRE of claim 9.

15. A method for treating a genetic disease or disorder in a subject by integrating a donor nucleic acid into a target nucleic acid of the subject, wherein the method comprises the steps of: (i) providing the vDRE according to claim 1, in a therapeutically effective amount, and a donor nucleic acid, optionally allowing the vDRE to be expressed, and (ii) allowing the vDRE to integrate the donor nucleic acid into the target nucleic acid.

16. A method for treating a genetic disease or disorder in a subject by exchanging a nucleic acid present in the genome of a subject or a cell, wherein the method comprises the steps of: (i) providing the vDRE according to claim 1, in a therapeutically effective amount, and a different nucleic acid that shall replace the nucleic acid in the genome of the subject or the cell, optionally allowing the vDRE to be expressed, and (ii) allowing the vDRE to exchange the nucleic acid in the genome of the subject or cell and insert the different nucleic acid.


SEQUENCE LISTING (31 sequences)

SEQ ID NO: 1 - DNA, 13 bp, synthetic construct
  source 1..13: mol_type = other DNA
acaccgggtcttc

SEQ ID NO: 2 - DNA, 13 bp, synthetic construct
  source 1..13: mol_type = other DNA
gaagacctgttta

SEQ ID NO: 3 - skipped

SEQ ID NO: 4 - DNA, 34 bp, synthetic construct
  source 1..34: mol_type = other DNA
ataacttcgtataacgtgatctatacgaagttat

SEQ ID NO: 5 - DNA, 34 bp, synthetic construct
  source 1..34: mol_type = other DNA
ataacttcgtatacagtataatatacgaagttat

SEQ ID NO: 6 - DNA, 34 bp, synthetic construct
  source 1..34: mol_type = other DNA
ataacttcgtataaaccggtctatacgaagttat

SEQ ID NO: 7 - DNA, 34 bp, synthetic construct
  source 1..34: mol_type = other DNA
ataacttcgtataatgtatgctatacgaagttat

SEQ ID NO: 8 - protein, 343 aa, P1 Bacteriophage
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 9-12 - skipped (4 entries)

SEQ ID NO: 13 - protein, 343 aa, synthetic construct
MSNLLTIHQNLPALPVDATSDEVRKNLMDMFRDRQAFSEHTWKMLLSVCRSWAAWCRLNNRKWFPAEPEDVRDYLLYLQARGLAVKTIQQHLGQLNMLHRRSGLPQPSDSNAVSLVMRRIRKENVDAGERAIQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQTGGWTNVNIVMNYIRNLDSETGAMVRLLEDGD

SEQ ID NO: 14 - protein, 343 aa, synthetic construct
MSNLLTVHKNLPALPVDATSDEVRKNLTDMFRDRQAFSEHTWEALLSVCRSWAAWCELNNRKWFPAEPEDVRDYLLHLQARGLAVNTVQQHLARLNMLHRRFGLPRPSDSNAVSLVMRRIRKENVDAGERAKQALAFERTDFDQVRSLMENSDRCQDIRNLAFLGIAYNTLLRIAEIARVRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTDVNIVMNYIRNLDSETGAMVRLLEDDD

SEQ ID NO: 15 - protein, 343 aa, synthetic construct
MSNLPTAHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAEQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNDVAAPSATSQLSTRALEGIFEATHRLIYGAKDDSGQRYLAWSGHSARVGAARDMARAGVSIPEIMQAGGWITVNSVMYYIRNLDSETGAMVRLLEDGD

SEQ ID NO: 16-18 - skipped (3 entries)

SEQ ID NO: 19 - DNA, 34 bp, synthetic construct
  source 1..34: mol_type = other DNA
ataacttcgtataaggtatgttatacgaagttat

SEQ ID NO: 20 - DNA, 34 bp, synthetic construct
  source 1..34: mol_type = other DNA
ataacttcgtataaggtatgctatacgaagttat

SEQ ID NO: 21 - DNA, 34 bp, synthetic construct
  source 1..34: mol_type = other DNA
ataacttcgtataagatatgctatacgaagttat

SEQ ID NO: 22 - DNA, 34 bp, synthetic construct
  source 1..34: mol_type = other DNA
ataacttcgtataaggtactctatacgaagttat

SEQ ID NO: 23-25 - skipped (3 entries)

SEQ ID NO: 26 - DNA, 29 bp, synthetic construct
  source 1..29: mol_type = other DNA
tttgaagaccccacccgcaagcttcacgg

SEQ ID NO: 27 - DNA, 28 bp, synthetic construct
  source 1..28: mol_type = other DNA
tttgaagacctaaacgcaccgagcacgc

SEQ ID NO: 28 - DNA, 56 bp, synthetic construct
  source 1..56: mol_type = other DNA
gtgactggagttcagacgtgtgctcttccgatctgtatgacatgtcgcgaaacacc

SEQ ID NO: 29 - DNA, 54 bp, synthetic construct
  source 1..54: mol_type = other DNA
acactctttccctacacgacgctcttccgatctctatgcataaacgcaccgagc

SEQ ID NO: 30 - DNA, 20 bp, synthetic construct
  source 1..20: mol_type = other DNA
gcgtcacactttgctatgcc

SEQ ID NO: 31 - DNA, 24 bp, synthetic construct
  source 1..24: mol_type = other DNA
gttcgccagttaatagtttgctgc